(19) United States
(12) Patent Application Publication
(10) Pub. No.: US 2008/0171066 A1
(43) Pub. Date: Jul. 17, 2008

(54) Listeriolysin-Containing Bacillus Spores as Antigen Delivery Agents

(76) Inventor: Simon Cutting, Egham (GB)

Correspondence Address: NIXON & VANDERHYE, PC, 901 NORTH GLEBE ROAD, 11TH FLOOR, ARLINGTON, VA, 22203, US

(21) Appl. No.: 11/884,529

(22) PCT Filed: Feb. 20, 2006
(86) PCT No.: PCT/GB2006/000582

§ 371 Date: Feb. 13, 2008

(30) Foreign Application Priority Data
Feb. 19, 2005 (GB) 0503509.2

(51) Int. Cl. A61K 39/07; C12N 1/20; A61P 37/00; C12N 15/74; A61K 35/74

(52) U.S. Cl. 424/246.1; 435/252.31; 424/93.2; 435/471

(57) ABSTRACT

The present invention is based on the provision of non-pathogenic Bacillus spores comprising: (i) a polynucleotide sequence encoding a phagosome membrane-rupturing agent; and (ii) a polynucleotide sequence encoding at least one further heterologous polypeptide. These may be used to deliver heterologous polypeptides to cells and in particular to phagocytic cells. Pharmaceutical compositions, vaccines and medicaments comprising the spores are provided and may be used for a variety of purposes including in immunisation and vaccination.

### FIELD OF THE INVENTION

The present invention relates to an improved delivery agent for delivering heterologous polypeptides to cells. The invention also relates to the use of the improved delivery agent in vaccines, pharmaceutical compositions and to methods for the production of the agent.

### BACKGROUND OF THE INVENTION

It is often desired to deliver chosen polypeptides, or polynucleotides encoding chosen polypeptides, to cells. For instance, it is often desired to deliver antigens to the cells of the immune system in order to elicit an immune response in a subject and hence to vaccinate a subject against pathogens or conditions such as cancer or allergy. It may also be desired to deliver polynucleotides encoding desired polypeptides so that the chosen polypeptide is expressed in, and can be subsequently harvested from, or has an effect on, the cell. It may also be desired to deliver polypeptides to label cells, to correct an underlying defect in the cell or even to kill a particular subset of cells.

In an effort to control infectious diseases in human and non-human animals, and in particular in mammals, the induction of a strong immune response is desired and hence the delivery of antigens to the cells of the immune system is an important goal. Protection against many infectious diseases is conferred by vaccinating a population against specific diseases to reduce the risk of infection by those diseases. Many vaccine delivery systems are effective at generating a humoral (antibody) response in an animal, but remain ineffective at controlling some of the most serious diseases for which vaccines do not currently exist (Shata et al. (2000) Molecular Medicine Today 6:66-71). Notable examples where the generation of a humoral response is not sufficient to control a disease include malaria and AIDS.

To combat these diseases, and many others, the induction of a cellular immune response against antigens from the pathogens is desirable. Specifically, the induction of an antigen-specific cytotoxic T lymphocyte (CTL) response is desirable. In a CTL response cytotoxic T lymphocytes are stimulated by an antigen presented on the surface of a target cell to destroy the cell. Typically the antigen is presented in response to infection of the target cell by a virus or other intracellular pathogen. Whilst most CTL responses are mediated by MHC class I-restricted CD8+ cytotoxic T lymphocytes, in some cases CD4+ cells can also kill cells. The CTL response is important in the control of diseases which are caused by pathogenic agents which can survive intracellularly, within the host cell cytoplasm, such as malaria, TB and HIV (Debin, A. et al (2002) Vaccine 20:2752-63; Schafer, R. et al. (1992) J Immunol 149:53-9).

The cellular response, like the humoral response, is part of the adaptive immune system which allows a human or non-human animal to acquire immunity to a particular disease. The adaptive immune system contains two major lymphocytes: B-cells and T-cells. The lymphocytes are originally “naïve” but upon contacting an antigen, for example from a pathogen, T-cells expressing a T-cell receptor (TCR) specific for epitopes in the antigen are selected. Thus, T-cells specific to that antigen express receptors that recognise (bind specifically to) the antigen and proliferate and undergo affinity maturation to produce T-cells with still higher affinity for the antigen. This process is called proliferation and maturation. The process results in memory cells (dormant “stand-by” antigen-specific cells), and active B- and T-cells. Active B-cells (or plasma cells) produce antigen-specific antibodies that circulate and capture the antigen.

Active T-cells consist of two types, T-helper cells which encourage B-cells to produce more antibodies against the antigen, and T-cytotoxic cells which find and kill infected cells showing fragments of the antigen. B- and T-cells communicate to one another and to the innate immune system (macrophages, natural killer cells etc.) through small secreted molecules called cytokines. Memory cells stay dormant and upon contacting with the specific antigen (for example, on re-infection), they quickly become active, proliferating and maturing into antigen-specific B-cells and T-cells which immediately are involved in controlling the antigen. The existence of memory cells therefore means a more rapid and effective immune response can be raised after subsequent exposure to an antigen and forms the basis of immunisation.

### SUMMARY OF THE INVENTION

The present invention provides non-pathogenic Bacilli for use in delivering heterologous polypeptides. In particular, the invention provides non-pathogenic Bacillus spores comprising:
(i) a polynucleotide sequence encoding a phagosome membrane-rupturing agent; and
(ii) a polynucleotide sequence encoding at least one further heterologous polypeptide.

The invention also provides:

a pharmaceutical composition comprising non-pathogenic Bacillus spores of the invention and a pharmaceutically acceptable carrier, diluent or excipient;

non-pathogenic Bacillus spores of the invention for use in a method for treatment of the human or animal body by therapy; and

use of non-pathogenic Bacillus spores of the invention in the manufacture of a medicament for use in the treatment or prevention of infection, autoimmunity, allergy or cancer.

In a further instance, also provided is a method for treating or preventing infection, autoimmunity, allergy or cancer, the method comprising administering to a human, or non-human animal, an effective amount of non-pathogenic Bacillus spores of the invention or a pharmaceutical composition of the invention.

The invention additionally provides a method of producing non-pathogenic Bacillus spores of the invention, the method comprising:

(i) transforming into vegetative cells of the Bacillus a polynucleotide sequence encoding:

(a) a phagosome membrane rupturing agent; and/or

(b) a further heterologous peptide,

wherein either both are transformed into the Bacillus or the Bacillus already comprises one of the sequences of (i) or (ii); and,

(ii) inducing or allowing the Bacillus to sporulate in order to produce spores.

The invention also provides vegetative cells which may be used for the production of the spores of the invention. Thus, the invention additionally provides for vegetative cells of a Bacillus comprising:
(i) a polynucleotide sequence encoding a phagosome membrane-rupturing agent; and
(ii) a polynucleotide sequence encoding at least one further heterologous polypeptide.

### BRIEF DESCRIPTION OF FIGURES

FIG. 1 is a schematic diagram showing the intracellular fate of a Bacillus of the invention ingested by a phagocytic cell. The spore in the depicted case expresses the particular phagosome membrane-rupturing agent LLO.

FIG. 2 depicts schematically three different methods for expressing phagosome membrane-rupturing agents in Bacillus spores and hence for inducing CTL responses.

FIG. 3 is the nucleotide sequence (SEQ ID NO:1) of the hylA gene of Listeria monocytogenes (Accession No: AF253320 from Listeria monocytogenes ATCC 9525). The hlyA gene encodes the phagosome membrane rupturing agent listeriolysin O (LLO);

FIG. 4 is the amino acid sequence (SEQ ID NO:2) of the phagosome membrane rupturing agent LLO (Accession No: AAF64524.1 from Listeria monocytogenes ATCC 9525).

FIG. 5A illustrates schematically strategies to insert the sequences encoding a heterologous polypeptide into the B. subtilis genome using the pDL242/pDL243 vectors.

FIG. 5B illustrates in more detail the site of insertion of a gene encoding a heterologous polypeptide or hlyA in the pDL242 or pDL243 vectors of FIG. 5A.

FIG. 5C illustrates schematically the insertion of cloned DNA at the thrC locus of the B. subtilis chromosome.

FIG. 5D illustrates schematically the insertion of cloned DNA at the amyE locus of the B. subtilis chromosome.

FIG. 6A is a simple representation of the expression of LLO in vegetative cells from the PrrnO-hlyA (or PrrnO-LLO) gene, the PrrnO-hlyA cassette illustrated is ready for insertion into pDG364 or pDG1664;

FIG. 6B is a simple representation of the expression of a heterologous polypeptide/antigen (Ag) in vegetative cells from the PrrnO-Ag genes, the PrrnO-Ag cassette illustrated is ready for insertion into pDG364 or pDG1664.

FIG. 7A is a schematic representation of an expression cassette of PrrnO-LLO at the thrC locus for LLO expression.

FIG. 7B is a schematic representation of an expression cassette of PrrnO-LLO at the amyE locus for LLO expression.

FIG. 8 illustrates the expression of LLO during vegetative cell growth.

FIG. 9A is a schematic representation of the PrrnO-lacZ expression cassette at the thrC locus (pDL242 mediated). This vector allows expression of β-galactosidase during vegetative cell growth.

FIG. 9B illustrates the induction of a CTL response in spleen cells of mice immunised by the oral route with B. subtilis spores expressing PrrnO-LLO carried at the amyE locus and PrrnO-LacZ carried at the thrC locus (closed circles) or PrrnO-LacZ only (open circles) or naïve mice (asterisks).

FIG. 10A is a schematic representation of an expression cassette (pDL242) carrying the Influenza Nucleoprotein (NP) fused to PrrnO. This vector allows expression of NP under vegetative cell growth.

FIG. 10B illustrates the induction of a CTL response by B. subtilis spores expressing PrrnO-LLO and PrrnO-NP administered by the nasal route.

FIG. 11 illustrates the induction of a CTL response by Influenza NP carried on the spore coat.

FIG. 12A is a schematic representation of an expression cassette (pDL242) carrying HIV tat fused to PrrnO.

FIG. 12B illustrates the induction of a CTL response by HIV tat.

FIG. 13 illustrates the proliferation of spores/vegetative cells in intestinal macrophages.

FIG. 14 illustrates the induction of IL-1α expression in macrophages infected with spores expressing LLO, and spores expressing LLO and the tetC antigen.

FIG. 15 illustrates the induction of IL-6 expression in macrophages infected with spores expressing LLO, and spores expressing LLO and the tetC antigen.

FIG. 16 illustrates that in macrophages infected with spores expressing LLO, and spores expressing LLO and the tetC antigen, there is no induction of TNFα expression.

FIG. 17 illustrates the induction of a CTL response by HIV tat, and compares tat alone; tat and LLO; and LLO and a tat/LLO fusion/chimeric protein.

### BRIEF DESCRIPTION OF SEQUENCES

SEQ ID No: 1 provides the nucleotide sequence of the hylA gene of Listeria monocytogenes (Accession No: AF253320 from Listeria monocytogenes ATCC 9525) which encodes the LLO protein.

SEQ ID No: 2 provides the amino acid sequence of the phagosome membrane rupturing agent LLO (Accession No: AAF64524.1 from Listeria monocytogenes ATCC 9525).

### DETAILED DESCRIPTION

Throughout the present specification and the accompanying claims the words “comprise” and “include” and variations such as “comprises”, “comprising”, “includes” and “including” are to be interpreted inclusively. That is, these words are intended to convey the possible inclusion of other elements or integers not specifically recited, where the context allows. In some cases, where specific constituents are recited, the embodiment may, for example, consist essentially of such constituents.

The present invention provides a non-pathogenic Bacillus for the delivery of heterologous polypeptides to target cells and in particular to phagocytic cells. The Bacillus comprises coding sequences for both a phagosome membrane-rupturing agent and at least one further heterologous polypeptide where the Bacillus is capable of expressing the membrane rupturing agent and the further heterologous polypeptide. The membrane-rupturing agent facilitates entry of the Bacillus into the cytoplasm of the target cell from the phagosome.

In a particularly preferred embodiment, the invention may be used to deliver antigenic heterologous polypeptides in order to stimulate an immune response. The Bacillus of the invention may in a preferred embodiment be used in vaccination. In an especially preferred instance the Bacillus is provided in spore form.

### Bacilli

The present invention provides a delivery agent in the form of a non-pathogenic Bacillus. Thus, the invention provides a non-pathogenic Bacillus comprising:
(i) a polynucleotide sequence encoding a phagosome membrane-rupturing agent; and
(ii) a polynucleotide sequence encoding at least one further heterologous polypeptide.

The Bacillus may be in the form of a vegetative cell or a spore. In a particularly preferred instance, the invention provides the Bacillus in spore form. Thus, in particular, the invention provides non-pathogenic Bacillus spores each comprising (i) and (ii).

The Bacillus is typically capable of being phagocytosed by a human or a non-human animal cell. In particular, the Bacillus is capable of being phagocytosed by any of the phagocytic cells mentioned herein, especially those of the immune system. The spores may, for instance, be arranged to germinate upon phagocytosis, or may, for instance, remain as a spore and in a dormant state following phagocytosis.

The Bacillus of the invention comprises a polynucleotide sequence encoding a heterologous polypeptide in addition to comprising the coding sequences for the heterologous membrane-rupturing agent. By heterologous it is intended that neither the heterologous coding sequences, nor the heterologous polypeptide which is the expression product, are naturally present in the delivery agent into which either is introduced.

Where the delivery agent is a spore, the heterologous polypeptide coding sequence is not normally in the spore or in the organism from which the spore is derived. Even if the Bacillus and the source of the heterologous polypeptide exchange genetic information, the heterologous coding sequences would normally not be found in the wild-type Bacillus in nature. Usually, the term heterologous will involve species of different genera as delivery agent and gene source. Thus, typically a polynucleotide sequence will have been introduced artificially that encodes the heterologous polypeptide and/or the phagosome membrane-rupturing agent.

Thus, by heterologous it is meant something that is non-native to the Bacillus. In the situation where the Bacillus is to be administered to an organism, the polynucleotide and polypeptide may be heterologous to that organism, originate from it or originate from the same species. In a preferred instance they may be heterologous to the organism as well.

The Bacillus of the invention comprises a coding sequence for a phagosome membrane-rupturing agent and can express the agent. A phagosome membrane-rupturing agent typically allows release of the phagocytosed Bacillus, and in particular spore, into the cytosol of the phagosome from the phagolysosome. This gives access to delivered antigens to the MHC I pathway and hence is particularly useful for eliciting an immune response against intracellular pathogens.

The Bacillus of the invention is non-pathogenic. Any suitable Bacillus may be employed. Illustrative examples include Bacillus alvei; Bacillus badius; Bacillus brevis; Bacillus cereus; Bacilluscoagulans; Bacillus fastidiosus; Bacilluslichenif'ormis; Bacillus jnycoides; Bacillus pasteurii; Bacillus sphaericus; Bacillus aneurinolyticus; Bacilluscarotarum; Bacillus flexus; Bacillus freudenreichi; Bacillus ynaeroide; Bacillus similibedius; Bacillus thiaminolyticus; Bacillus subtilis; Bacillus pumilus; Bacillus vallismortis; Bacillusbengalicus; Bacillus flexus; and Bacillus licheniformis. An appropriate Bacillus may, for example, be produced by introducing the necessary sequences into any of the preceding Bacilli. In a particularly preferred instance, the Bacillus is Bacillus subtilis. The strain may be PY79 a prototrophic spo⁺ derivative of the type strain 168 (Youngman et al., 1984, Plasmid 12:1-9) or such a strain with any of the modifications discussed herein.

In one instance, the spore or vegetative cell is derived from a non-Bacillus anthracis bacterium species. In another instance, the Bacillus is a non-pathogenic Bacillus anthracis species.

In one instance, the Bacillus employed may be non-pathogenic, but originate from a pathogenic strain of Bacillus which has been modified to render it non-pathogenic. Any pathogenic strain may be so modified including Bacillus anthracis. Mutagenesis may be employed to render the strain non-pathogenic and for instance homologous recombination and gene targeting may be used to inactivate chosen genes to render the strain non-pathogenic. Such strains will be incapable of spontaneous reversion to their pathogenic form.

The Bacillus of the invention may be in spore or vegetative cell form and in particular when being administered to a subject or to a population of cells is in spore form. Typically, a spore is a small, usually single-celled reproductive body that is highly resistant to desiccation and heat, normally it is capable of growing into a new organism, however it may be modified such that it cannot develop into a new organism. Spores are produced by certain bacteria (in which case they are endospores), fungi, algae, and non-flowering plants (in which case they are exospores). Bacillus spores are employed in the invention.

The invention also provides a non-pathogenic Bacillus that has been genetically modified to encode a phagosome membrane rupturing agent and at least one further heterologous polypeptide.

The Bacillus of the invention may be phagocytosed and in particular may be phagocytosed by cells of the immune system. The cell of a human or non-human animal which phagocytoses a Bacillus delivery agent according to the invention may be any cell with the ability to phagocytose and in particular may be a leukocyte, a neutrophil, a monocyte, a macrophage or a dendritic cell. The cell may be, for instance, any phagocytic immune cell. Thus, in particular, a Bacillus of the invention may be phagocytosed by any of such cells and such phagocytosis will result in delivery of the desired heterologous polypeptide to the phagocyte. The Bacillus may have already expressed the further heterologous polypeptide prior to phagocytosis, or may express, or continue to express the heterologous polypeptide in the phagocyte. Expression may occur in the phagosome or once the Bacillus has escaped the phagosome. Similarly, the rupturing agent may, for instance, be expressed prior to administration or after phagocytosis.

### Heterologous Polypeptides

Preferably a Bacillus delivery agent according to the invention is typically arranged to deliver a heterologous polypeptide expressed from a heterologous coding sequence into a cell of a host human or non-human animal or population of cells. The Bacillus comprises coding sequences encoding a heterologous membrane-rupturing agent and also at least one further heterologous polypeptide. In some instances, the entire heterologous gene for the heterologous polypeptide or a heterologous protein may be present, in others it may not be. In some cases, the heterologous polypeptide may be a protein, in others it may be a shorter polypeptide such as for instance, from 5 to 100, preferably from 10 to 75 and even more preferably from 25 to 50 residues in length.

The polynucleotide sequence encoding the further heterologous polypeptide may, in some instances, encode a protein, or fragment of a protein, including ones normally expressed in nature by a pathogen. In particular, the further heterologous polypeptide expressed from the polynucleotide may be one from a pathogen. The pathogen may, for instance, be a virus, bacterium, parasite, protozoan, fungus, or prion. In one preferred embodiment, the heterologous polypeptide coding sequences encodes a protein or polypeptide from a pathogen such as a virus or other pathogenic agent that can survive intracellularly in a host cell.

The heterologous polypeptide and its encoding sequences may be a natural or a synthetic sequence. The heterologous coding sequences may comprise sequences which do not occur naturally. In some instances, the heterologous polypeptide may be encoded by a polynucleotide sequence where only those sequences encoding the heterologous polypeptide are heterologous. For instance, the promoter and/or other regulatory elements may originate from the Bacillus itself, or at least from another Bacillus strain. The regulatory sequences and elements for expression may originate from other heterologous sources to the coding sequences and may, for instance, be ones used in the art for expression in Bacilli. In other instances, the entire gene encoding the heterologous polypeptide and the membrane-rupturing agent may be heterologous and may originate from the same source.

The membrane rupturing agent and the further heterologous polypeptide may be expressed as a fusion with other sequences such as those originating from the Bacillus itself. The additional sequences may be N or C terminal or may be present at both termini. The heterologous polypeptide may, in one instance, be expressed as a fusion or chimera with the phagosome membrane-rupturing agent. In many embodiments it will not be so fused.

In some instances, a Bacillus may comprise a polynucleotide sequence or sequences encoding more than one heterologous polypeptide for instance, two, three, four or more heterologous polypeptides, aside from the heterologous phagosome membrane-rupturing agent, may be encoded. The phagosome membrane-rupturing agent itself is a heterologous polypeptide and the Bacillus has the ability to express at least one other heterologous polypeptide sequence aside from the rupturing agent or as a fusion with the agent.

Expression of the further heterologous polypeptide from the heterologous coding sequences in a host cell, or release of the heterologous polypeptide, in many embodiments preferably elicits an immune response to the heterologous polypeptide in the human or non-human animal of which the host cell is a part. Preferably the immune response elicited by the antigen is a cellular response and in particular a CTL response. In some instances the response is predominately a CTL response. In other instances, an antibody response may be elicited and in others both a CTL and an antibody response may be elicited. The responses elicited may be against any of the antigens mentioned herein.

The heterologous polypeptide may be, or comprise, a cytokine such as, for instance, and interleukin or a colony-stimulating factor and in particular IL-1α, IL-12, IL-18, and GM-CSF. The polypeptide may be an enzyme, structural protein, hormone, antibody or an adjuvant polypeptide. The heterologous polypeptide may be a functional fragment of any of the preceding. The heterologous polypeptide may be an interferon, fragment or variant thereof e.g IFN-α,β or γ

### Antigens

The further heterologous polypeptide may, in one particularly preferred instance, comprise a polynucleotide which encodes an antigen, immunogenic fragment thereof or immunogenic variant of either. The antigen may comprise, or indeed be as small as, a single eptiope or alternatively may comprise a plurality of epitopes. The antigen may comprise multiple copies of the same epitope or of a different epitope. The heterologous polypeptide may comprise an immunogenic fragment of an antigen or an immunogenic variant of the antigen or fragment.

The antigen, fragment or variant may be an antigen from a pathogen, a tumour or cancer antigen, an autoimmune antigen or an allergen antigen. In a preferred instance, the antigen may be one from a pathogen and in particular a viral, bacterial, parasitic or fungal pathogen antigen. In a preferred instance, the antigen may be a viral antigen, an immunogenic fragment thereof or an immunogenic variant of the antigen or fragment.

As the heterologous polypeptide may comprise an antigen, fragment thereof or variant of either the Bacillus may be used as, or in, a vaccine for the treatment or prevention of a number of conditions including, but not limited to, cancer, allergies, toxicity and infection by a pathogen such as, but not limited to, fungus, viruses including Human Papilloma Viruses (HPV), HIV, HSV2/HSV1, influenza virus (types A, B and C), Polio virus, RSV virus, Rhinoviruses, Rotaviruses, Hepatitis A virus, Norwalk Virus Group, Enteroviruses, Astroviruses, Measles virus, Para Influenza virus, Mumps virus, Varicella-Zoster virus, Cytomegalovirus, Epstein-Barr virus, Adenoviruses, Rubella virus, Human T-cell Lymphoma type I virus (HTLV-I), Hepatitis B virus (HBV), Hepatitis C virus (HCV), Hepatitis D virus, Pox virus, Marburg and Ebola; bacteria including Mycobacterium tuberculosis, Chlamydia, N. gonorrhoea, Shigella, Salmonella, Vibrio Cholera, Treponema pallidua, Pseudomonas, Bordetella pertussis, Brucella, Franciscella tulorensis, Helicobacter pylori, Leptospria interrogaus, Legionella pnumophila, Yersinia pestis, Streptococcus (types A and B), Pneumococcus, Meningococcus, Hemophilus influenza (type b), Toxoplama gondii, Complybacteriosis, Moraxella catarrhalis, Donovanosis, and Actinomycosis; fungal pathogens including Candidiasis and Aspergillosis; parasitic pathogens including Taenia, Flukes, Roundworms, Amebiasis, Giardiasis, Cryptosporidium, Schistosoma, Pneumocystis carinii, Trichomoniasis and Trichinosis. The further heterologous polypeptide(s) may comprise an antigen, fragment or variant from such pathogens. In a preferred instance, the antigen may be a Bacillus anthracis antigen and in particular encode the Protective Antigen of Bacillus anthracis or an immunogenic fragment or immunogenic variant.

The Bacillus may also be used to provide a suitable immune response against numerous veterinary diseases, such as Foot and Mouth diseases, Coronavirus, Pasteurella multocida, Helicobacter, Strongylus vulgaris, Actinobacillus pleuropneumonia, Bovine viral diarrhea virus (BVDV), Klebsiella pneumoniae, E. coli, Bordetella pertussis, Bordetella parapertussis and Bordetella brochiseptica.

A Bacillus may encode a polypeptide for treating or preventing a cancer. In a particularly preferred embodiment a construct of the invention may encode a tumour antigen. Examples of tumour associated antigens include, but are not limited to, cancer-testes antigens such as members of the MAGE family (MAGE 1, 2, 3 etc), NY-ESO-1 and SSX-2, differentiation antigens such as tyrosinase, gp100, PSA, Her-2 and CEA, mutated self antigens and viral tumour antigns such as E6 and/or E7 from oncogenic HPV types. Further examples of particular tumour antigens include MART-1, Melan-A, p97, beta-HCG, GaINAc, MAGE-1, MAGE-2, MAGE-4, MAGE-12, MUC1, MUC2, MUC3, MUC4, MUC18, CEA, DDC, P1A, EpCam, melanoma antigen gp75, Hker 8, high molecular weight melanoma antigen, K19, Tyr1, Tyr2, members of the pMel 17 gene family, c-Met, PSM (prostate mucin antigen), PSMA (prostate specific membrane antigen), prostate secretary protein, alpha-fetoprotein, CA125, CA19.9, TAG-72, BRCA-1 and BRCA-2 antigen. Examples of particular cancers that the antigen may be include those from cancers of the lung, prostate, breast, colon, ovary, testes, bowel, melanoma, a lymphoma and a leukaemia. The Bacillus may be used to deliver the heterologous polypeptide to cancer cells themselves or preferably to cells of the immune system to stimulate an immune response.

In one preferred instance the Bacillus may comprise a polynucleotide encoding an antigen, immunogenic fragment thereof or an immunogenic variant of either from a virus and in particular from a member of the adenoviridae (including for instance a human adenovirus), herpesviridae (including for instance HSV-1, HSV-2, EBV, CMV and VZV), papovaviridae (including for instance HPV), poxyiridae (including for instance smallpox and vaccinia), parvoviridae (including for instance parvovirus B19), reoviridae (including for instance a rotavirus), coronaviridae (including for instance SARS), flaviviridae (including for instance yellow fever, West Nile virus, dengue, hepatitis C and tick-borne encephalitis), picornaviridae (including polio, rhinovirus, and hepatitis A), togaviridae (including for instance rubella virus), filoviridae (including for instance Marburg and Ebola), paramyxoviridae (including for instance a parainfluenza virus, respiratory syncitial virus, mumps and measles), rhabdoviridae (including for instance rabies virus), bunyaviridae (including for instance Hantaan virus), orthomyxoviridae (including for instance influenza A, B and C viruses), retroviridae (including for instance HIV and HTLV) and hepadnaviridae (including for instance hepatitis B). In one instance the antigen may be from a pathogen responsible for a veterinary disease and in particular may be from a viral pathogen, including, for instance, a Reovirus (such as African Horse sickness or Bluetongue virus) and Herpes viruses (including equine herpes). The antigen may be one from Foot and Mouth Disease virus. In a further preferred instance the antigen may be from a Tick borne encephalitis virus, dengue virus, SARS, West Nile virus and Hantaan virus.

In another preferred case the antigen may be from a retroviradae (e.g. HTLV-I; HTLV-11; or HIV-1 (also known as HTLV-111, LAV, ARV, hTLR, etc.)). In particular from HIV and in particular the isolates HIVIllb, HIVSF2, HTVLAV, HIVLAI, HIVMN; HIV-1CM235, HIV-1; or HIV-2. In a particularly preferred embodiment, the antigen may be a human immunodeficiency virus (HIV) antigen. Examples of preferred HIV antigens include, for example, gp120, gp 160 gp41, gag antigens such as p24gag and p55gag, as well as proteins derived from the pol, env, tat, vif, rev, nef, vpr, vpu or LTR regions of HIV. In a particularly preferred case the antigen may be HIV gp120 or a portion of HIV gp120. The antigen may be from an immunodeficiency virus, and may, for example, be from SIV or a feline immunodeficiency virus.

In one instance, the antigen may be one secreted or released by a pathogen including any of those mentioned herein. Examples of preferred bacterial antigens include: the Shigella sonnei form 1 antigen; the F1 antigen of Yersinia pestis; antigens from Neisseria meningititidis and in particular those encoded by the GNA33, GNA2001, GNA1220 and GNA1946 genes; the O-antigen of V. cholerae Inaba strain 569B; protective antigens of enterotoxigenic E. coli, such as fimbrial antigens including colonisation factor antigens, in particular CFA/I, CFA/II, and CFA/IV and the nontoxic B-subunit of the heat-labile toxin; pertactin of Bordetella pertussis, adenylate cyclase-hemolysin of B. pertussis; fragment C of tetanus toxin of Clostridium tetani and the LT (heat labile enterotoxin) and ST (heat stable toxin) antigens. Immunogenic fragments and variants may also be encoded. In a preferred instance, the antigen may be a toxin antigen and in particular a tetanus toxin. The antigen may be Tetanus Toxin fragment C, an immunogenic fragment thereof or an immunogenic variant of either.

In one instance, the heterologous protein gene encodes a protein normally expressed in nature by a pathogen. In another the pathogen is one that can survive intracellularly in a host cell. In a preferred instance, the pathogen is an immunodeficiency virus or an influenza virus.

Thus, the encoded polypeptide may be an antigen, an immunogenic fragment thereof or an immunogenic variant thereof. The fragment or variant may, for instance, have any of the levels of homology, proportion of the length of the original antigen, and functionality specified herein and in particular ability to give rise to an immune response. The antigen may be the full length of the naturally occurring, or known, protein or it may be shorter or indeed longer. In some instances the sequence expressed may be a short fragment of an antigen or a variant thereof, such as from 5 to 100, preferably from 10 to 50, more preferably from 10 to 25 and even more preferably from 10 to 20 amino acids in length. In some instances, the encoding sequence of the nucleic acid construct may have been modified to optimize expression. For instance, codon usage may be modified to that typical of the Bacillus. A consensus Kozak sequence for the subject may also be substituted for the naturally occurring sequence around the start codon. Such modifications may have been made to any of the heterologous coding sequences discussed herein.

In instances where the antigen is an influenza antigen, the influenza antigen may, for instance, be an influenza NP (nucleoprotein/nucleocapsid protein), HA (hemagglutinin), NA (neuraminidase), M1, M2, PB1, PB2, PA, NS1 and/or NS2 antigens or may be a fragment or variant of such antigens. In one preferred embodiment, the encoded antigen may be HA, NA and/or M2 influenza antigen or a fragment or a variant of such antigens. In an especially preferred instance, the encoded antigen may be an HA or an NA antigen or a fragment or variant of such antigens and in particular an HA antigen or a fragment or variant of such an antigen.

In one preferred embodiment the antigen may be from the H5N1 strain of influenza or be an immunogenic fragment thereof or a variant of either which retains immunogenicity. The antigen, fragment or variant may be from one of the antigens used in annual influenza vaccines and in some cases the Bacillus may express all three of the annually employed influenza antigens. Alternatively either two or three different Bacilli expressing between them the three antigens such as, for instance, one strain expressing two antigens and the other one or each strain expressing an individual influenza antigen may be employed.

The Bacilli of the invention may be provided, or used, in combination with each other to provide multivalent vaccines, such as divalent, trivalent, tetravalent vaccines and so on, including such combinations of any of the antigens mentioned herein.

In some cases the antigen may be an antigen from a prion. In particular, the antigen may be one from the causative agent of kuru, Creutzfeldt-Jakob disease (CJD), scrapie, transmissible mink encephalopathy and chronic wasting diseases, or from a prion associated with a spongiform encephalopathy, particularly BSE. The antigen may be from the prion responsible for familial fatal insomnia.

In some cases the antigen may be from a parasitic pathogens including, for example, one from the genera Plasmodium, Chtamydia, Trypanosome, Giardia, Boophilus, Babesia, Entamoeba, Eimeria, Leishmania, Schistosome, Brugia, Fascida, Dirofilaria, Wuchereria and Onchocerea. Examples of preferred antigens from parasitic pathogens to be expressed as the heterologous antigen include the circumsporozoite antigens of Plasmodium species, such as the circumsporozoite antigen of P. bergerii or the circumsporozoite antigen of P. falciparum; the merozoite surface antigen of Plasmodium species; the galactose specific lectin of Entamoeba histolytica; gp63 of Leishmania species; paramyosin of Brugia malayi; the triose-phosphate isomerase of Schistosoma mansoni; the secreted globin-like protein of Trichostrongylus colubriformis; the glutathione-S-transferases of Frasciola hepatica, Schistosoma bovis and S. japonicum; and KLH of Schistosoma bovis and S. japonicum.

In a particularly preferred embodiment of the invention the heterologous polypeptide comprises an antigen, fragment or variant from, or derived from, an intracellular pathogen. Thus, in one instance the antigen may be from Mycobacterium tuberculosis (Tuberculosis), Mycobacterium leprae (Leprosy), Listeria monocytogenes (Listeriosis), Salmonella typhi (Typhoid Fever), Shigella dysenteriae (Bacillary dysentery), Yersinia pestis (Plague), Brucella species (Brucellosis), Legionella pneumophila (Pneumonia), Rickettsiae (Typhus; Rocky Mountain Spotted Fever), Chlamydia (Chlamydia; Trachoma), or Bacillus anthracis (anthrax). Bacillus anthracis antigens are particularly preferred.

Other preferred pathogens include Streptococcus pyogenes, Staphylococcus aureus, Bacillus anthracis, Streptococcus pneumoniae, Klebsiella pneumoniae, Haemophilus influenzae, Pseudomonas aeruginosa, Salmonella typhi, Salmonella typhimurium, Listeria monocytogenes, Clostridium perfringens, Yersinia pestis, Yersinia enterocoliticai, Mycobacterium tuberculosis, Legionella pneumophilia, Neisseria gonorrhoeae, Rickettsia, Chlamydia, Brucella abortus, Treponema pallidum, Escherichia coli, and Leishmania. Bartonella grahamii, Chlamydia trachomatis, Cryptococcus neoformans, Erhlichia chaffeensis, Francisella tularensis, Histoplasma capsulatum, Legionella pneumophila, Leishmania mexicana, Shigella Flexner, Toxoplasma gondii, Haemophilus somnus.

The antigen may be an auto-antigen. In particular, the antigen may an antigen associated with an autoimmune disease. Auto-antigens include those associated with autoimmune diseases such as multiple sclerosis, insulin-dependent type 1 diabetes mellitus, systemic lupus erythematosus (SLE) and rheumatoid arthritis. The antigen may be one associated with, Sjorgrens syndrome, myotis, scleroderma or Raynaud's syndrome. Further examples of auto-immune disorders that the antigen may be associated with include ulcerative colitis, Crohns' disease, inflammatory bowel disorder, autoimmune liver disease, or autoimmune thyroiditis. Examples of specific autoantigens include insulin, glutamate decarboxylase 65 (GAD65), heat shock protein 60 (HSP60), myelin basic protein (MBP), myelin oligodendrocyte protein (MOG), proteolipid protein (PLP), and collagen type II.

In some cases the antigen may be an allergen. The allergenic antigen may be any suitable antigen from an antigen. For example, the allergen may be from Ambrosia artemisiifolia, Ambrosia trifida, Artemisia vulgaris, Helianthus annuus, Mercurialis annua, Chenopodium album, Salsola kali, Parietaria judaica, Parietaria officinalis, Cynodon dactylon, Dactylis glomerata, Festuca pratensis, Holcus lanatus, Lolium perenne, Phalaris aquatica, Phleum pratense, Poa pratensis or Sorghum halepense. The allergen antigen may be from a tree, such as, for example, from Phoenix dactylifera, Betula verrucosa, Carpinus betulus, Castanea sativa, Corylus avellana, Quercus alba, Fraxinus excelsior, Ligustrum vulgare, Olea europea, Syringa vulgaris, Plantago lanceolata, Cryptomeria japonica, Cupressus arizonica, Juniperus oxycedrus, Juniperus virginiana, or Juniperus sabinoides. In some cases the antigen may be from an antigen from a mite such as, for example, from Acarus siro, Blomia tropicalis, Dermatophagoides farinae, Dermatophagoides microceras, Dermatophagoides pteronyssinus, Euroglyphus maynei, Glycyphagus domesticus, Lepidoglyphus destructor or Tyrophagus putrescentiae.

The allergen antigen may be from an animal such as, for example, from a domestic or agricultural animal. Examples of allergens from animals include those from cattle, horses, dogs, cats and rodents (e.g from rat, mouse, hamster, or guinea pig). In some cases the antigen may be from a food allergen and in others it may be from insect. Viruses, including any of those mentioned herein are also preferred intracellular pathogens.

Expression or release of the heterologous protein or polypeptide into a host cell may induce the expression of cytokines and interleukins in a human or non-human animal of which the host cell is a part. The cytokines and interleukins induced may include IL-α, IL-12, IL-18, and GM-CSF. The induction of cytokines and interleukins may be part of the CTL response. In one instance, the heterologous polypeptide delivered may be a cytokine including any of the preceding cytokines. In other instances the heterologous polypeptide delivered may be an adjuvant polypeptide. The heterologous polypeptide may serve as a marker for instance it may be β-galactosidase.

### Fragments & Variants

In the present invention naturally occurring, or known, sequences may, in some instances, be employed. However, in some cases fragments of naturally occurring or known sequences or variants of either may be employed. In particular, fragments or variants having a particular functionality may be employed.

Thus, in the case of the further heterologous polypeptide the coding sequences may themselves be, or comprise, a fragment or variant of a naturally occurring sequence and/or encode a polypeptide that comprises a fragment or variant of a naturally occurring sequence. The fragment or variant will typically retain at least some functionality such as at least one function, and in some cases all of the functions of the original polypeptide. For instance, in the situation where the fragment or variant is derived from an antigen, they will be immunogenic and preferably be able to elicit an immune response against the original antigen.

In situations where a fragment or variant is obtained or derived from an enzyme, they will typically retain some enzymatic activity unless such activity is unnecessary for the intended purpose, such as eliciting an immune response. Similarly, fragments and variants of cytokines and adjuvant polypeptides will retain at least some functionality in their ability to act as a cytokine or act as an adjuvant.

The Bacilli of the invention comprise a coding sequence of a phagosome membrane-rupturing agent. Again, the natural coding sequences may, in some instances, be employed. However, in others functional fragments or variants may be employed both at the level of the coding sequences and/or at the level of the encoded polypeptide. The fragments and variants will retain at least some functionality. That is they will allow escape of a Bacillus to the cytosol of a phagosome and bring about membrane rupture of the phagosome.

A variant or fragment may be less, or more, active than the wild-type polypeptide and may, for instance, have at least 5%, preferably at least 10%, more preferably at least 25%, more preferably at least 50% and even more preferably at least 75% of the activity of the wild type polypeptide and in some cases may be at least as active as the wild type polypeptide. In some instances, the polypeptide may have at least an additional 50%, at least double or at least treble the activity of the original polypeptide.

Any suitable assay may be used to compare the activity of fragments and variants in comparison to the wild type sequence. For instance, the immune response generated may be compared using any of the assays mentioned herein.

At the amino acid level, a variant may, for instance, have amino acid substitutions in comparison to the sequence of the polypeptide it is derived from. For example, it may have from 1, 2, 3 or more substitutions such as from 5 to 10, 10 to 20, 20 to 30 or more amino acid substitutions. In some instances, 1% or more, 2% or more, 5% or more or 10% or more of the amino acid residues of the wild-type, or original polypeptide may have been substituted. A variant may have the same or less than any of the preceding numbers of substitutions. The variant may have, in addition or alternatively, such numbers of amino acids deleted or inserted into it in comparison to the original sequence. The deletions, insertions or substitutions may be closely grouped or spread out.

The amino acid changes may be conservative substitutions, for example according to the following Table. Amino acids in the same block in the second column and preferably in the same line in the third column may be substituted for each other. The substitutions may be non-conservative.

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

A variant may have a particular level of sequence identity to the wild type polypeptide. For instance, a polypeptide may have, at least 50%, preferably at least 65% identity, preferably at least 80% or at least 90% and particularly preferably at least 95%, at least 97% or at least 99% identity with the amino acid sequence of the wild type sequence. As indicated above, the variant will retain the particular desired functionality such as the ability to give rise to an immune response in the case of an antigen.

Such levels of sequence identity may, for instance, be over the whole of the variant, over at least 20, preferably at least 50, more preferably at least 75, and even more preferably over at least 100 amino acids. For instance, in the case of the membrane rupturing agent the level of sequence identity may be over at least 50, preferably at least 100, more preferably over at least 250 and even more preferably over at least 400 amino acid residues and particularly preferably over the entire length of the variant and/or the wild type polypeptide.

In some instances fragments of wild type sequences may be employed or indeed fragments of variants. Such fragments will preferably retain a desired functionality. Thus, in the case of an antigen, the fragment will still be able to give rise to an immune response against the original antigen or in the case of a rupturing agent display at least some membrane rupturing activity.

A fragment, may, for instance, be at least 5%, preferably at least 10%, more preferably at least 25%, more preferably at least 50% and still more preferably at least 75% of the length of the wild type sequence. In some instances, it may be at least 85%, preferably at least 95%, more preferably at least 97% and even more preferably at least 99% of the length of the wild type polypeptide. In some instances, the fragment may be equal than or less than such lengths.

In some instances, for example where the heterologous coding sequence is a fragment of an antigen the fragment may be quite short in comparison to the wild type sequence, for instance as a minimum a single epitope cable of giving rise to an immune response. The fragment may, for instance, be less than 75, preferably less than 50 and even more preferably less than 25 amino acid residues in length. In other instances, such as where the fragment is a fragment of a membrane rupturing agent the fragment may be longer. For instance, it may be at least 200, preferably at least 300, even more preferably at least 400 amino acid residues in length.

Fragments and variants of nucleotide sequences may also be employed. Such fragments and variants may, for instance, have any of the specified lengths, degrees of sequence identity or homology and so on specified above or may encode such a polypeptide. The polynucleotide variant or fragment may comprise polynucleotide insertions, substitutions, or deletions, including any of the numbers specified above in relation to amino acid sequences. For instance, a sequence may have at least 1, 5, 10, 15, 20, 25 or more substitutions, insertions or deletions or have equal or less numbers of such modifications. Variant sequences may be such that they encode the same polypeptide, but differ at the nucleotide level because of the degeneracy of the genetic code.

In one preferred instance, the membrane-rupturing agent employed and the coding sequences utilised will be those of the LLO polypeptide and hylA gene whose sequence is provided in SEQ ID Nos 1 and 2. In others, fragments and variants of such sequences may be employed, particularly those employing any of the levels of sequence homology or identity, overall size in comparison to the original sequence and so on discussed herein.

In some instances, a polynucleotide or polypeptide employed in the invention may comprise such fragments or variants as discussed above. The polynucleotides and polypeptides may comprise additional elements as discussed elsewhere. Thus, the levels of sequence identity, homology and so on to the naturally occurring, or known sequence, may be over the length of the relevant polypeptide or polynucleotide derived from the naturally occurring sequences. Alternatively, they may be measured over the length of the entire polynucleotide or polypeptide.

A variety of programs may be used to calculate percentage homology and sequence identity. The UWGCG Package provides the BESTFIT program which can be used to calculate homology (for example used on its default settings) (Devereux et al (1984) Nucleic Acids Research 12, p387-395). The PILEUP and BLAST algorithms can be used to calculate homology or line up sequences (typically on their default settings), for example as described in Altschul S. F. (1993) J Mol Evol 36:290-300; Altschul, S, F et al (1990) J Mol Biol 215:403-10. Software for performing BLAST analyses is publicly available through the National Centre for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighborhood word score threshold (Altschul et al, supra). These initial neighborhood word hits act as seeds for initiating searches to find HSPs containing them. The word hits are extended in both directions along each sequence for as far as the cumulative alignment score can be increased. Extensions for the word hits in each direction are halted when the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

### Phagosome Membrane-Rupturing Agents

The phagosome membrane-rupturing agent may be typically any protein or polypeptide which has membrane lytic properties. Many proteins which are amphipathic with a positive charge bias can target and bind membranes, and may at a high enough concentration cause a membrane to rupture. Examples of amphipatic proteins include, but are not limited to, Gramicidin S (an ampipathic decapeptide), Streptolysin O (SLO), Influenza Haemagglutin (HA) peptides, Pefringolysin O (PFO), PI-PLC (phosphatidylcholine (PI)-specific phospholipase C (from L. monocytogenes), delta-Lysin or δ-Lysin (from Staphylococcus aureus), Defensins, Magainins and Cecropins. Funtional fragments and variants of such rupturing agents may also be employed.

Preferably the phagosome membrane-rupturing agent is a haemolysin. The haemolysin may be listeriolysin 0 (LLO), a Listeria monocytogenes haemolysin. Functional fragments or variants of such rupturing agents may be employed. Variants of functional fragments may be employed where they retain the ability to act as membrane rupturing agents. The LLO polypeptide of SEQ ID No: 2, a functional fragment thereof or a functional variant of either may be employed. The coding sequence of SEQ ID No: 1, a fragment therefore encoding a functional agent or a variant or either encoding a functional agent may be employed.

Expression of the phagosome membrane-rupturing agent in a phagocytosed delivery agent preferably causes the phagosome membrane to rupture. Preferably the phagosome membrane-rupturing agent is secreted from the delivery agent into the phagosome and then causes the phagosome membrane to rupture. The rupturing agent may therefore be expressed with a single peptide including any or those discussed herein. Alternatively, the agent may be expressed on the surface of the Bacillus, particularly in the spore form, to bring about rupture. The rupturing agent may have been expressed prior to phagocytosis and/or administration or alternatively after such steps.

### Expression of Coding Sequences

The Bacillus of the invention comprises:
(i) a polynucleotide sequence encoding a phagosome membrane-rupturing agent; and
(ii) a polynucleotide sequence encoding at least one further heterologous polypeptide.

The Bacillus will be able to express (i) and (ii). Thus, the coding sequences will be operably linked to the sequences to allow their expression and in particular to an appropriate promoter. The two may be expressed from separate promoters or the same promoter. In situations where they are expressed from the same promoter they may be expressed as a fusion or a chimera and optionally subsequently cleaved. Appropriate cleavage sequences may be present.

In one preferred instance, the Bacillus includes one or more constructs including the phagosome membrane-rupturing agent coding sequences and/or the coding sequences for the at least one further heterologous polypeptide. The one or more further heterologous polypeptide coding sequence may be on the same, or on a different, vector construct to each other and to the coding sequences encoding the phagosome membrane-rupturing agent. The one or more constructs may also include other sequences or genetic elements required for expression of the coding sequences. Such sequences or genetic elements are well known to the man skilled in the art, and may include one or more enhancer elements, upstream activation sequence and/or other regulatory control element. The constructs may comprise the entire gene for the membrane rupturing agent and/or further heterologous coding sequence. They may therefore be gene constructs.

The promoter or promoters employed for expression of the coding sequences may, for instance, be inducible, strong or modified. The promoter(s) may be homologous or heterologous to the gene to be expressed. The promoter may be a vegetative cell promoter or a spore promoter. The promoter may be one active on germination. The promoters may originate from the Bacillus itself or from another Bacillus type in some embodiments. A preferred promoter for use in expression is the PrrnO promoter. Functional fragments and variants of the PrrnO promoter may be also employed.

Expression of the heterologous polypeptide and/or the phagosome membrane-rupturing agent may be inducible, constitutive or may only occur at a particular time in the life cycle of the Bacillus. In one preferred instance one, or both, and in particular the heterologous polypeptide, may be expressed on germination of the spore and in particular will be so expressed in the phagosome or following escape from the phagosome.

The constructs may comprise DNA or cDNA. The construct may comprise a polynucleotide analogue. The constructs employed may in some instances use the pDL242 or pDL243 vectors as a backbone.

The one or more constructs encoding the further heterologous polypeptide and/or the phagosome membrane-rupturing agent may be inserted into a chromosome of the delivery agent. Thus, the Bacillus, in some instances, does not comprise any extrachromosomal constructs or at least none encoding one or both of the phagosome membrane-rupturing agent or the heterologous polypeptide.

Insertion into the chromosome may be by recombination. Homologous recombination and gene targeting may, in particular, be used to introduce the chosen polynucleotides into the chromosome of the Bacillus. Gene targeting or mutagenesis may be used to also inactivate chosen coding sequences, for instance to help render the Bacillus non-pathogenic.

Such techniques may be used to insert chosen sequences into, or outside of, the endogenous genes of the chromosome. In preferred instances, sequences may be targeted into the thrC and/or amyE loci of the Bacillus. The selection criteria described herein for identifying homologous recombinants may be employed. In some instances the targeting construct may not comprise the promoter or other elements for the expression of the coding sequences and those of endogenous genes may be used via the targeting. In other cases the targeting construct may comprise the elements necessary for expression. The phagosome membrane-rupturing agent coding sequences and those encoding the further heterologous polypeptide may be introduced via single or multiple targeting steps.

In one instance, the inserted coding sequences/DNA does not disrupt the normal function of any of the genes on the chromosome. Alternatively, the genes or gene constructs encoding the further heterologous protein or polypeptide and/or the phagosome membrane-rupturing agent may be inserted into a vector, such as a bacteriophage, in the delivery agent. If the delivery agent is a bacterial spore the bacteriophage may, for instance, be SPβ. The Bacillus is also provided as vegetative cells that can sporulate to provide such spores. Alternatively, the gene or gene constructs encoding the further heterologous polypeptide and/or the phagosome membrane-rupturing agent may be inserted into and expressed from an autonomously replicating vector, such as a plasmid in the delivery agent.

If the delivery agent is a spore, the genes or gene constructs are preferably inserted into a vegetative cell of the spore-forming organism after which the organism is induced to form spores, the spores formed will therefore also include the genes or gene constructs. All of the Bacilli mentioned herein are provided in both spore and vegetative cell form.

The invention also provides method of producing non-pathogenic Bacillus spores of the invention, the method comprising:

(i) transforming into vegetative cells of the Bacillus a polynucleotide sequence encoding:
(a) a phagosome membrane rupturing agent; and/or
(b) a further heterologous peptide,

wherein either both are transformed into the Bacillus or the Bacillus already comprises one of the sequences of (i) or (ii); and

(ii) inducing or allowing the Bacillus to sporulate in order to produce spores.

The method may involve any of the constructs discussed herein and in particular gene targeting constructs. An additional selection or secreening step or steps may be performed to identify those desired clones and particularly desired homologous recombinants. The invention also provides a method of producing the Bacillus which simply comprises step (i) without sporulation in order to produced vegetative cells of the invention which can then, if desired, be used to produce spores.

Preferably the one or more heterologous polypeptide coding sequence referred to with reference to the present invention comprise sufficient genetic code to encode an expression a heterologous protein which is functional for the purpose intended. For example, if the delivery means is to be used as a vaccine or for immunisation the further heterologous protein expressed by the heterologous protein gene is preferably able to elicit an immune response. In other instances a functional enzyme, structural polypeptide, cytokine or adjuvant polypeptide may be delivered.

Preferably the phagosome membrane-rupturing agent gene or coding sequence referred to with reference to the present invention comprises sufficient genetic code to encode on expression a protein that is functional and can cause the phagosome membrane to rupture. Assays may be used to determine whether a particular polypeptide has such functionality including any of those discussed herein.

In some embodiments of the invention, the entire genes which comprise the coding sequences for the further heterologous polypeptide and/or the rupturing agent will be heterologous, in other embodiments only the coding sequences may be. In some instances, the constructs employed will be gene constructs and comprise all the sequences necessary for expression, in others such sequences may be provided by the Bacillus particularly following insertion into the chromosome.

Expression of the one or more genes encoding the further heterologous protein and/or the phagosome membrane-rupturing agent may occur in the delivery agent before or after it is phagocytosed. Expression may occur before, during or after sporulation.

In a preferred embodiment the rupturing agent and/or the further heterologous polypeptide are secreted. Preferably the phagosome membrane-rupturing agent encoded by the phagosome membrane rupturing agent gene or coding sequences includes a signal sequence. The signal sequence may cause the phagosome membrane-rupturing agent to be secreted from a phagocytosed delivery agent. The signal sequence is preferably an N-terminal signal sequence. The further heterologous protein may also be fused to a signal sequence.

The signal sequence may be hydrophobic. The signal sequence may allow secretion of heterologous proteins and polypeptides from a phagocytosed Bacillus. Any suitable signal sequence functional in the Bacillus may be employed and in particular the signal sequence of amino acids 1 to 28 of SEQ ID No:2 or a functional fragment thereof, or a functional variant may be employed. The polypeptide may also comprise a cleavage sequence to allow removal of the signal sequence, such as a protease cleavage sequence and in particular a cleavage sequence for signal sequence peptidase I.

The further heterologous protein or polypeptide may be arranged such that it is expressed on the surface of the delivery agent for instance on the surface of the spore or vegetative cells and in particular on the surface of the spore. The heterologous protein or polypeptide may be fused to a coat protein of the delivery agent thereby causing expression of the heterologous protein on the surface of the delivery agent. If the delivery agent is a spore of Bacillus subtilis the spore protein fused to the heterologous protein may be CotA, CotB, CotC, CotD, CotE and/or CotF. The phagosome rupturing agent may also be expressed in any of the preceding ways. Functional fragments or variants of coat proteins which allow for expression on the spore surface may be employed.

In some embodiments heterologous polypeptides may include cleavage sequence, such as for a proteases, to allow release or activation of particular elements. Cleavage sequences for proteases found in the phagosome and/or the cytosol of the phagocyte may, for instance, be employed. Heterologous polypeptides may include tags, such as, for instance, tags to allow purification. Examples include a His tag.

The further heterologous protein or polypeptide may be arranged such that on expression it is fused, as a chimera, with the phagosome membrane-rupturing agent. Fusion of the further heterologous protein to the phagosome membrane-rupturing agent may be at the N or C terminus of the heterologous protein or polypeptide. The phagosome rupturing agent may also be expressed in any of the preceding ways.

### Subjects

The Bacilli of the present invention may be administered to a variety of subjects. The Bacillus and various entities provided by the invention may be administered to any suitable subject. The host cell may be the cell of a human or non-human animal, or a population of cells. Thus the subject may be human or non-human. Preferably a non-human animal is a mammal.

The subject is generally a vertebrate subject. By “vertebrate subject” is meant any member of the subphylum cordata, particularly mammals, including, without limitation, humans and other primates, as well as rodents, such as mice, guinea pigs and rats.

In one preferred instance the subject is human. The subject may be a non-human animal. The non-human animal may be a domestic animal or an agriculturally important animal. For instance, the subjects may be cattle, pigs, horses, sheep or goats, they may be sports animals such as horses and dogs. The animal may be a domestic pet such as a dog or cat. The animal may be a monkey such as a non-human primate such as a chimpanzee, gorilla or orangutan.

The term subject does not denote a particular age. Thus, both adult and newborn individuals are intended to be covered. In one embodiment the subject is susceptible to or at risk from the relevant disease. For example, the subject may have been exposed, or will be in a region where there is a risk of exposure, to a particular antigen and in particular a pathogen.

### Compositions, Vaccines, Medicaments, Formulation and Administration

The Bacilli of the invention may be used to deliver therapeutic polypeptides and the invention therefore provides a range of therapeutic products and methods.

The invention also provides a pharmaceutical composition comprising non-pathogenic Bacillus spores of the invention and a pharmaceutically acceptable carrier, diluent or excipient. In one instance, the composition may be a vaccine. The invention also provides a vaccine comprising a Bacillus of the invention and in particular non-pathogenic Bacillus spores of the invention. The vaccine may additionally comprise a pharmaceutically acceptable carrier, diluent or excipient.

The invention also provides for non-pathogenic Bacillus spores of the invention for use in a method for treatment of the human or animal body by therapy. The method may be to treat, prevent or ameliorate any of the conditions mentioned herein. In a particularly preferred instance, the method may be a method of vaccination or immunisation. In one instance, the vaccination or immunisation is to treat, prevent or ameliorate an infection, an autoimmune condition, allergy or cancer. The method may be for treating, preventing or ameliorating the effect of a toxin.

The invention also provides for the use of non-pathogenic Bacillus spores of the invention in the manufacture of a medicament for use in the treatment or prevention of infection, autoimmunity, allergy or cancer. In addition, also provided is a method for treating or preventing infection, autoimmunity, allergy or cancer, the method comprising administering to a human, or non-human animal, an effective amount of non-pathogenic Bacillus spores or a pharmaceutical composition of the invention.

The various compositions, vaccines and other substances of the invention may be formulated using any suitable method. Formulation with standard pharmaceutically acceptable carriers and/or excipients may be carried out using routine methods in the pharmaceutical art. For example, the Bacillus may be in physiological saline or water. The exact nature of a formulation will depend upon several factors including the particular Bacillus to be administered and the desired route of administration.

Suitable types of formulation are fully described in Remington's Pharmaceutical Sciences, 19^{th} Edition, Mack Publishing Company, Eastern Pennsylvania, USA, the disclosure of which is included herein of its entirety by way of reference.

The substances may be administered by enteral or parenteral routes such as via oral, buccal, anal, pulmonary, intravenous, intra-arterial, intramuscular, intraperitoneal, topical or other appropriate administration routes. The substances may in some cases be administered to sites characterised by the presence of phagocytes. In preferred instances, the compositions of the invention may, for instance be administered orally, rectally, vaginally or nasally. In a particularly preferred instance compositions of the invention may be administered orally or nasally and in particular orally. In particular, the Bacillus will be in spore form for such administration.

The pharmaceutical composition, vaccine or protein delivery means may, for instance, be administered orally as a liquid, a paste, a tablet or a capsule. Intranasal administration may suitably be in the form of a fine powder or aerosol nasal spray or in particular cases in the form of modified Dischaler® or Turbohaler®. Rectal administration may suitably be via a suppository. When the composition is in the form of a powder, it may preferably be provided in an air-tight container such as a sachet or bottle, or inhaler.

A pharmaceutical composition, vaccine or means for delivering a protein according to the invention may be in preferred instances administered mucosally or parenterally. Mucosal administration may be administrated by any suitable route, particularly by an oral, nasal, rectal and/or a vaginal route. A vaccine delivered at the mucosal surfaces will particularly effective in combating those diseases which infect via the mucosal route.

A vaccine delivered by mucosal administration is preferably taken up by mucosal immune tissue. For an orally delivered vaccine this may be by the gut associated lymphoid tissue (GALT), and more specifically may be by the Peyer's Patches (PP) of the small intestine that are rich in antigen presenting cells, such as dendritic cells (DCs) and/or the mesenteric lymphoid nodes. For a nasally delivered vaccine, the spore may be taken up by the nasal associated lymphoid tissue (NALT). Any cells with the capacity for phagocytosis may be targeted and an appropriate route chosen.

A vaccine according to the invention has the advantage that it may be used to vaccinate for conditions where conventional vaccination methods have been unsuccessful, such as, for instance, HIV. A vaccine according to the invention may give protective immunity to infection caused by any of the pathogens mentioned herein and in a preferred instance to an immunodeficiency virus, such as HIV, or another viral agent. Preferably a vaccine according to the invention induces a cellular immune response or a predominately cellular response. A vaccine may also induce a humoral response.

According to another aspect, the invention provides a pharmaceutical composition or a vaccine comprising two or more delivery agents, wherein a first delivery agent has been genetically modified to encode a phagosome membrane-rupturing agent, and a second delivery agent has been genetically modified to encode a heterologous protein. The delivery agent is preferably a Bacillus, including any of those described herein, apart from the fact that the Bacillus does not encode both the rupturing agent and the heterologous polypeptide and instead the two are collectively encoded by two or more Bacilli. In one embodiment the invention provides a combination of two different Bacillus, one comprising a polynucleotide sequences encoding a phagosome membrane rupturing agent and a separate Bacillus comprising a polynucleotide sequence encoding a heterologous polypeptide. Such combinations may be employed in any of the methods described herein.

Thus, the invention provides a pharmaceutical composition or a vaccine comprising two or more delivery agents, wherein a first delivery agent has been genetically modified to encode a phagosome membrane-rupturing agent, and a second delivery has been genetically modified to encode a heterologous protein. The invention also provides a pharmaceutical composition or a vaccine comprising two or more delivery agents, wherein a first delivery agent comprises a phagosome membrane-rupturing agent protein and the second delivery agent contains a further at least one heterologous protein.

It will be appreciated that the first and second delivery agents may be administered simultaneously, either in the same or different formulations, or sequentially. When there is sequential administration, the delay in administering the second delivery should not be such as to lose the beneficial effect of the combination, that is the lysing of the phagosome to release the heterologous protein into the cytosol of the cell. In a preferred aspect of the invention the first delivery agent and the second delivery agent are administered in a combined formulation.

According to a yet further aspect, the invention provides the use of a delivery agent or composition according to the invention in the preparation or manufacture of a medicament for use in the treatment or prevention of a medical condition. Preferably wherein the medical condition is a pathogen infection. The medical condition may also be a cancer or tumour, allergy or an auto immune disease and may be the specific disease linked to any of the antigens mentioned herein. The medicament may be a vaccine.

According to another aspect, the invention provides a method of medical treatment, which method comprises the steps of administering an effective amount of a delivery agent or composition according to the invention to a human or non-human animal. Preferably the delivery agent is phagocytosed by a cell in the intestinal, respiratory or reproductive tract of the human or non-human animal. Once phagocytosed the delivery agent may secrete the phagosome membrane rupturing agent into the phagosome of the host cell. The phagosome membrane-rupturing agent may cause the phagosome to rupture allowing exposure of the heterologous protein or polypeptide to the host cell cytosol. The presence of the heterologous protein or polypeptide in the host cell cytosol may elicit an immune response in, or to, the host. Preferably the immune response is cell mediated and in particular is a CTL response.

According to another aspect, the invention provides a method for eliciting an immune response in a human or non-human animal comprising administering to the human or non-human animal an effective amount of a delivery agent or composition according to the invention. Preferably the immune response is a cell mediated and in particular is a CTL response.

Examples of excipients which may be present in the various compositions of the invention include a diluent (e.g. a starch or cellulose derivative, a sugar derivative such as sucrose, lactose or dextrose), a stabilizer (e.g. a hygroscopic component such as silica or maltodextrin), a binder, buffer (e.g. a phosphate buffer), a lubricant (e.g. magnesium stearate), coating agent, preservative, emulsifier, dye, flavouring, and/or suspension agent. Suitable excipients are well known to a person of skill in the art.

The various products of the invention may comprise a carrier or excipient which may be a solvent, dispersion medium, coating, isotonic or absorption delaying agent, sweetener or the like. These include any and all solvents, dispersion media, coatings, isotonic and absorption delaying agents, sweeteners and the like. Suitable carriers may be prepared from a wide range of materials including, but not limited to, diluents, binders and adhesives, lubricants, disintegrants, colouring agents, bulking agents, flavouring agents, sweetening agents and miscellaneous materials such as buffers and adsorbents that may be needed in order to prepare a particular dosage form.

For example, the solid oral forms may contain, together with the active compound, diluents such as lactose, dextrose, saccharose, cellulose, corn starch or potato starch; lubricants such as silica, talc, stearic acid, magnesium or calcium stearate and/or polyethylene glycols; binding agents such as starches, arabic gums, gelatin, methylcellulose, carboxymethylcellulose, or polyvinyl pyrrolidone; disintegrating agents such as starch, alginic acid, alginates or sodium starch glycolate; effervescing mixtures; dyestuffs, sweeteners; wetting agents such as lecithin, polysorbates, lauryl sulphates. Such preparations may be manufactured in known manners, for example by means of mixing, granulating, tabletting, sugar coating, or film-coating processes.

Liquid dispersions for oral administration may be syrups, emulsions and suspensions. The syrups may contain as carrier, for example, saccharose or saccharose with glycerol and/or mannitol and/or sorbitol. In particular, a syrup for diabetic patients can contain as carriers only products, for example sorbitol, which do not metabolise to glucose or which only metabolise a very small amount to glucose. The suspensions and the emulsions may contain as carrier, for example, a natural gum, agar, sodium alginate, pectin, methylcellulose, carboxymethylcellulose or polyvinyl alcohol.

In one instance, the compositions of the invention are administered to achieve a daily intake of between about 10⁴ to about 10¹⁵ colony forming units (CFU) of the Bacillus according to the invention, more preferably from 10⁶ to 10¹⁴ cfu, more preferably from 10⁸ to 10¹³ cfu, even more preferably from 10⁹ to 10¹⁰ cfu.

In some instances, an adjuvant may also be administered simultaneously, sequentially or separately to the Bacillus and in particular in the same composition as the Bacillus. Examples of adjuvants that may be employed include cytokines. Certain cytokines, for example TRANCE, fit-3L, and CD40L, enhance the immunostimulatory capacity of antigen presenting cells and may be employed. Non-limiting examples of cytokines which may be used alone or in combination include, interleukin-2 (IL-2), stem cell factor (SCF), interleukin 3 (IL-3), interleukin 6 (IL-6), interleukin 12 (IL-12), G-CSF, granulocyte macrophage-colony stimulating factor (GM-CSF), interleukin-1 alpha (IL-1 a), interleukin-11 (IL-11), MIP-1a, leukemia inhibitory factor (LIF), c-kit ligand, thrombopoietin (TPO), CD40 ligand (CD40L), tumor necrosis factor-related activation-induced cytokine (TRANCE) and flt3 ligand (flt-3L). Such adjuvants, functional fragments or functional variants of either may be encoded by the Bacillus in some embodiments. Such Bacilli may also encode an antigen, bE administered with an antigen or a Bacillus encoding an antigen.

Further examples of adjuvants which may be effective include but are not limited to: aluminium hydroxide, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion.

A substance of the invention may, for instance, be given in a single dose schedule, or preferably in a multiple dose schedule. The dosage regimen will also, at least in part, be determined by the need of the individual and be dependent upon the judgement of the practitioner. In the case of immunisation and vaccination boosting may be used to enhance the protective immune response generated.

### Methods

According to another aspect the invention provides the Bacillus of the invention or a composition according to the invention for use as a vaccine. The present invention therefore also provides for the use of the Bacilli in a method of vaccination. A delivery agent according to the invention may therefore be used to vaccinate or immunise a host human or non-human animal or cell population. Preferably, if the heterologous polypeptide expressed or contained within the delivery agent is a polypeptide normally expressed by a pathogen, the host will be vaccinated or immunised against the pathogen from which the heterologous polypeptide is derived. The heterologous polypeptide expressed or contained by the delivery agent may give the host a degree of immunity to one or more forms of cancer, the delivery agent may therefore be used as a cancer vaccine. The agent may be used to treat, prevent and/or ameliorate the various conditions and infections mentioned herein.

Preferably, once a Bacillus according to the invention has been administered to a subject (a human or non-human animal, or a population of cells) the delivery agent will be phagocytosed by a host cell. Preferably the host cell is a phagocyte such as a leukocyte, neutrophil, monocyte, macrophage or dendritic cell. The delivery agent may then be located in a phagosome in the host cell. Once in the phagosome the delivery agent may express the phagosome membrane-rupturing agent. The phagosome membrane-rupturing agent may then be secreted from the delivery agent into the phagosome, once in the phagosome the phagosome membrane-rupturing agent may cause the phagosomal membrane to rupture.

Rupture of the phagosomal membrane may release the delivery agent into the host cell cytosol. The heterologous protein or polypeptide may be expressed in the delivery agent before or after rupture of the phagosomal membrane. Indeed, the polypeptide may have been expressed in the Bacillus prior to administration to the subject in some instances.

After rupture of the phagosomal membrane the heterologous polypeptide may then be released into the cytosol of the host cell. The heterologous polypeptide may, for example, be released into the cytosol by secretion from the delivery agent, expression on the surface of the delivery agent or release from the delivery agent following lysis of the delivery agent. Once in the cytosol the heterologous polypeptide may, for instance, be processed via a Type/Class I pathway leading to MHC presentation of the heterologous protein on the host cell surface. This presentation may, in a preferred instance induce an antigen (heterologous protein)-specific cytotoxic T lymphocyte (CTL) response. This response may destroy the host cell presenting the heterologous protein (antigen) but may also provide the subject with a degree of immunity to subsequent infection by a pathogen expressing the same heterologous protein (antigen). Thus, the invention can be used to vaccinate subjects to induce the desired immunity. This process may also be used to introduce a heterologous polypeptide into a host cell that does not elicit an immune response for instances where the heterologous polypeptide is not an antigen and/or is not heterologous to the subject.

### Further Aspects

The invention also provides for delivering heterologous polypetides to phagocytes in vitro. The invention provides a cell or population of cells comprising phagocytes that have phagocytosed a Bacillus of the invention. Such cells may be used in ex vivo therapy and be subsequently administered to the same or different subject to the one they are recovered from, typically the same subject.

According to a further aspect the invention provides a means to deliver a polypeptide or protein to a host cell and in particular the cytosol of a host cell comprising a Bacillus according to the invention and a suitable carrier, diluent or excipient. This aspect of the invention may provide a tool for targeting molecules to the cytosol of a target cell.

The use of a delivery agent according to the invention as a pharmaceutical composition, a vaccine or a means to deliver a protein to the cytosol of a cell has the advantage that is removes the need for injections and the problems associated with needles in developing countries. Oral administration in particular is a quick and simple administration route no involving injection. In addition, if the delivery agent is a spore it will be stable and resistant to heat and desiccation making shipping and storage easier than for other forms of pharmaceutical composition, vaccine or protein delivery means. The production of spores is also relatively easy and can be done at a low cost making the production of vaccines economically viable particularly for developing countries. Furthermore as the Bacillus, including the spores, is a non-pathogenic organism the Bacillus safer than some more conventional methods.

The use of the Bacillus, particularly in spore from as the delivery agent in a vaccine has the further advantage that an immune response can be elicited at the mucosal membrane. This makes the vaccination more effective against mucosal pathogens.

According to a further aspect, the present invention provides a method of producing a genetically modified spore for use as a delivery agent according to the invention, which method comprises the steps;
producing one or more gene constructs encoding a phagosome membrane rupturing agent and a further at least one heterologous polypeptide;
using said at least one genetic construct to transform a vegetative mother cell;
inducing said transformed mother cell to sporulate; and
isolating the resulting genetically modified spores.

The invention also provides a method of producing non-pathogenic Bacillus spores of the invention, the method comprising:

(i) transforming into vegetative cells of the Bacillus a polynucleotide sequence encoding:
(a) a phagosome membrane rupturing agent; and/or
(b) a further heterologous peptide,
wherein either both are transformed into the Bacillus or the Bacillus already comprises one of the sequences of (i) or (ii); and
   (ii) inducing or allowing the Bacillus to sporulate in order to produce spores.

In another embodiment, the invention also provides a method of producing a genetically modified spore for use as a delivery agent of the invention, which method comprises the steps:
producing one or more gene constructs encoding at a phagosome membrane rupturing agent and a further at least one heterologous protein;
using said at least one genetic construct to transform a vegetative mother cell;
inducing said transformed mother cell to sporulate; and

isolating the resulting genetically modified spores.

In another instance, the present invention provides a method of vaccination, which method comprises the steps of:
orally or intra-nasally or rectally administering a Bacillus or composition according to invention to a human or non-human animal in need of vaccination;
said genetically modified delivery agent eliciting an immune response in the human or non-human animal.

According to another aspect the present invention provides a method of vaccinating or immunising a human or non-human animal comprising administering to the animal an effective amount of a delivery agent or composition according to the invention.

The invention also provides a method of vaccinating or immunising a human or non-human animal or cell population comprising administering to the animal an effective amount of a delivery agent of the invention.

The invention also provides a means to deliver a protein to the cytosol of a host cell comprising a delivery agent of the invention and a suitable carrier, diluent or excipient.

It will be appreciated that preferred features of the invention discussed with reference to only some aspects of the invention can equally be applied to all aspects of the invention.

The invention will now be described merely by way of example with reference to the accompanying figures, methods and Examples.

### Further-Discussion of the Figures

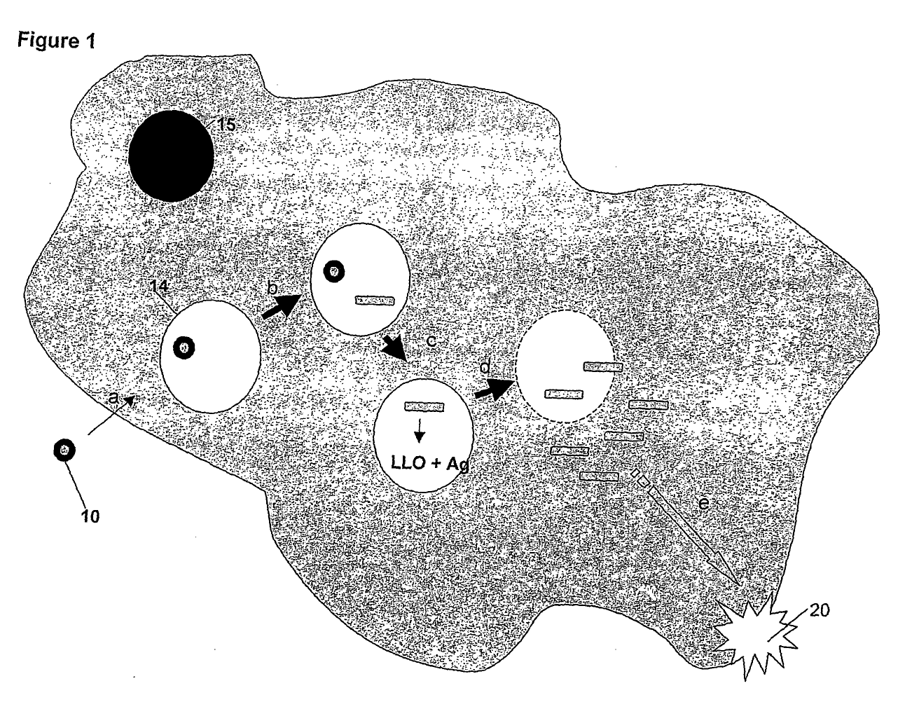

FIG. 1 shows schematically that the co-expression of phagosome membrane-rupturing agents, such as in the illustrated case LLO, and a heterologous antigen enhances the CTL response in a cell. A bacterial spore 10 is first internalized (step a) within a phagosome 14 in a cell 12. This step is termed phagocytosis. The cell 12 also comprises a nucleus 15. Studies have shown that the spores of most Bacillus spp. ingested by a cell in this way will germinate into vegetative cells (step b) and within 5-10 hours could be completely destroyed (Duc, L. et al.(2004) Vaccine 22:1873-1885). However, germinated Bacillus species that express, upon germination, phagosome membrane rupturing agents such as listerioloysin (LLO) (step c) are able to rupture the phagosomal membrane (step d) to enter the host cell cytoplasm and proliferate for a short period of time to allow more effective expression of the heterologous antigen and also entry of the expressed polypeptide into the MHC I antigen presentation pathway.

Typically, intracellular expression within the cytosol (or cytoplasm) of the heterologous polypeptide is followed by MHC Class I processing of the heterologous polypeptide (step e) and the activation of a CD8+ CTL response 20. In cases where the heterologous polypeptide is heterologous to the Bacillus, but is a native protein of the subject, the polypeptide may serve other functions when it reaches the cytosol. For instance, the polypeptide delivered in the invention may be a cytokine, adjuvant polypeptide, enzyme, or structural polypeptide.

In the embodiment depicted in FIG. 1, the phagosome membrane rupturing agent listerioloysin (LLO) is secreted from the germinating spore or vegetative cells into the phagosome to cause the rupturing of the phagosomal membrane. Once ruptured, the further heterologous polypeptide expressed by the vegetative cells following germination of the spore, is released into to the cytosol of the host cell. The heterologous polypeptide may also be expressed on the surface of the spore or vegetative cell, secreted into the cytosol from the vegetative cell or released into the cytosol following lysis of the vegetative cell.

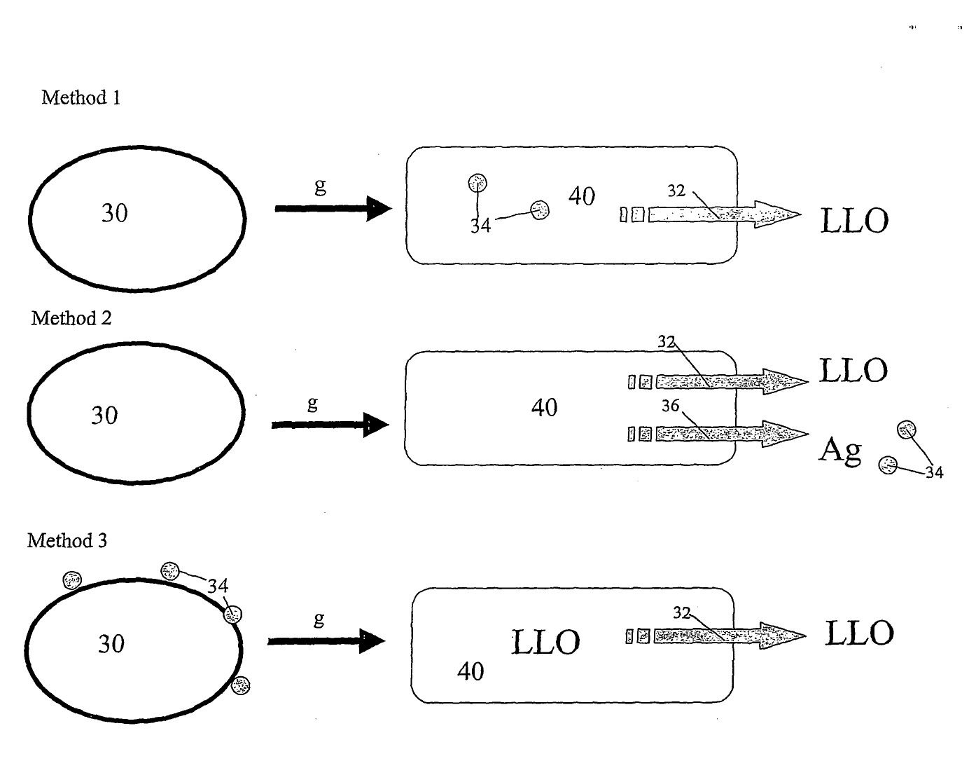

FIG. 2 depicts schematically three different methods for expressing phagosome rupturing agents such as LLO and a further heterologous polypeptide in the vegetative cells 40 of germinating Bacillus spores 30 and inducing a CTL response. In each case the specific phagosome membrane rupturing agent LLO (listeriolysin O) is expressed from a PrrnO-LLO cassette carried stably on the Bacillus chromosome. The LLO carries a membrane secretion sequence.

The three mechanisms depicted may be employed and are not limited to any specific construct or polypeptide. They are:

(1) Heterologous Polypeptide Expressed in Germinating Spore and Rupturing Agent Secreted from the Germinating Spore

Spore 30 is used for delivery of the further heterologous polypeptide gene and LLO gene into a host cell. Once in the host the spore 30 germinates (step g) and further the heterologous polypeptide and LLO are expressed within the vegetative cell 40. LLO is secreted 32 from the vegetative cell 40 because of its N-terminal signal sequence leading to the rupture of the phagosomal membrane and entry of the vegetative cell into the cytoplasm. The heterologous polypeptide 34 is expressed within the germinated spore/vegetative cell 40 but is not secreted. As the vegetative cell 40 replicates the heterologous polypeptide is released. Class I processing of the released heterologous polypeptide 34 will lead to presentation of peptides from the heterologous polypeptide 34 on the host cell surface and in the situation where the polypeptide is heterologous to the subject a CTL response.

(2) Secretion of Both Heterologous Polypeptide and Rupturing Agent from The Germinating Spore

Spore 30 is used for delivery of the further heterologous polypeptide gene and the LLO gene into a host cell. Once in the host the spore germinates (step g) and the heterologous polypeptide 34 and LLO are expressed within the vegetative cell 40. LLO is secreted 32 from the vegetative cell 40 because of its N-terminal signal sequence leading to rupture of the phagosomal membrane and entry of the vegetative cell 40 into the cytoplasm. The heterologous polypeptide 34 is also expressed within the germinated spore/vegetative cell 40 and is secreted 36 due to an N-terminal signal sequence fused to the N-terminus of the heterologous polypeptide 34. As the heterologous polypeptide is now in the cytosol class I processing of the secreted heterologous polypeptide 34 will occur and in embodiments where the polypeptide is both heterologous to the Bacillus and the subject a CTL response will result.

A variation of this method would be secretion of a membrane rupturing agent-further heterologous polypeptide sequence chimera. The further heterologous protein is fused to the C terminus or internally to rupturing agent sequences. Secretion of the chimera full-length ruptures the phagosomal membrane while in the situation where the heterologous polypeptide comprises an antigen the chimera generates a CTL response to the antigen.

### (3) Heterologous Polypeptide Expressed on the Spore Surface and the Phagosome Rupturing Agent Expressed in Germinating Spore or Secreted From it

Spore 30 carries the further heterologous polypeptide 34 presented on the spore surface fused to a spore coat protein. LLO is produced in the germinating (step g) spore 30. The uptake of the spore 30 into a host cell activates spore germination. Germination leads to the cracking of the spore coat and release of heterologous polypeptide 34 fused to the spore coat. The rupturing agent, in this case LLO, is secreted 32 from the germinating spore/vegetative cell 40 because of its N-terminal signal sequence leading to rupture of the phagosomal membrane and entry of the vegetative cell 40 into the cytoplasm. Rupture of the phagosomal membrane allows spore coat and its associated heterologous polypeptide 34 to enter the cytoplasm and be processed by a class I pathway, and hence where the heterologous polypeptide is heterologous to both the Bacillus and the subject a CTL response results.

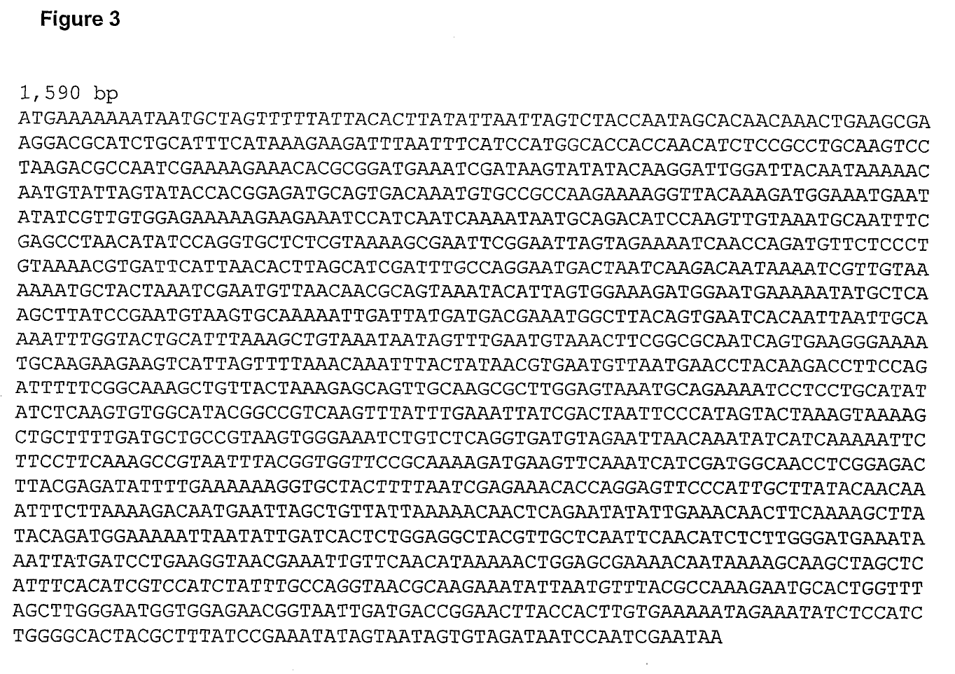

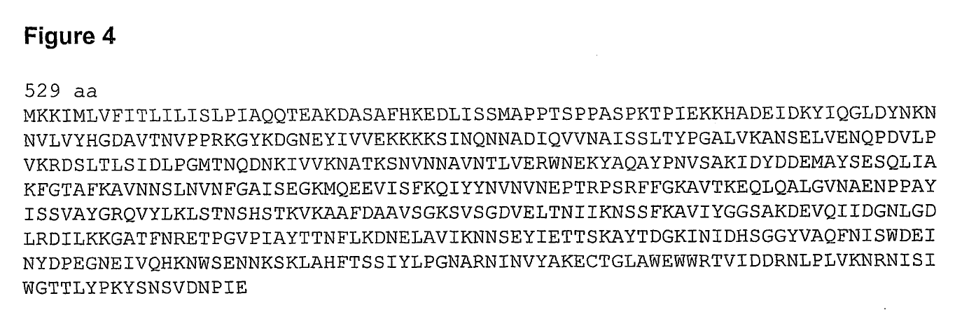

FIGS. 3 and 4 show the DNA and amino acid sequence respectively for the hlyA gene of Listeria monocytogenes. The hylA gene (FIG. 3) encodes listeriolysin 0 (LLO; FIG. 4). LLO (FIG. 4) carries an N-terminal hydrophobic signal sequence (residues 1-28) that allows secretion across a bacterial membrane, such as the membrane of a vegetative cell produced upon germination of a bacterial spore. Such a secretion signal, a functional fragment of it, or a functional variant of either may be employed in the present invention to give rise to secretion of the further heterologous polypeptide/ the rupturing agent or both.

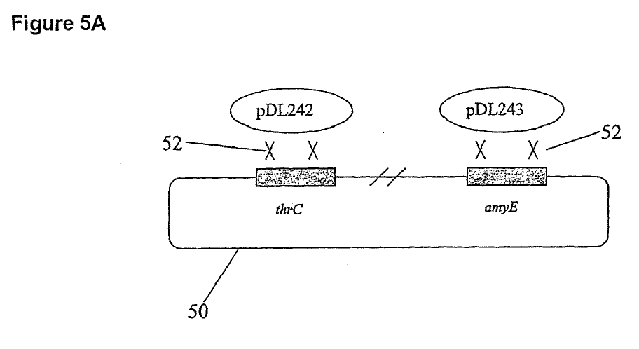

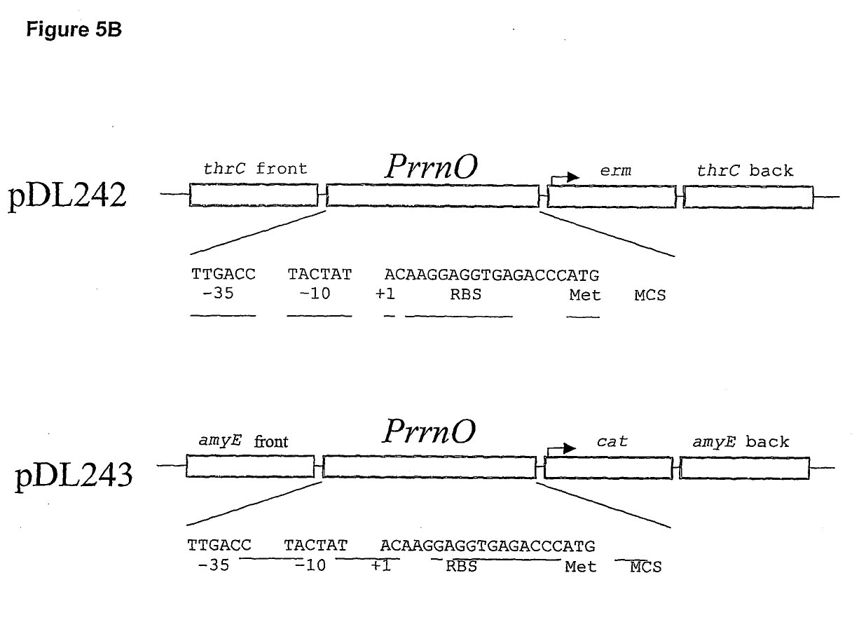

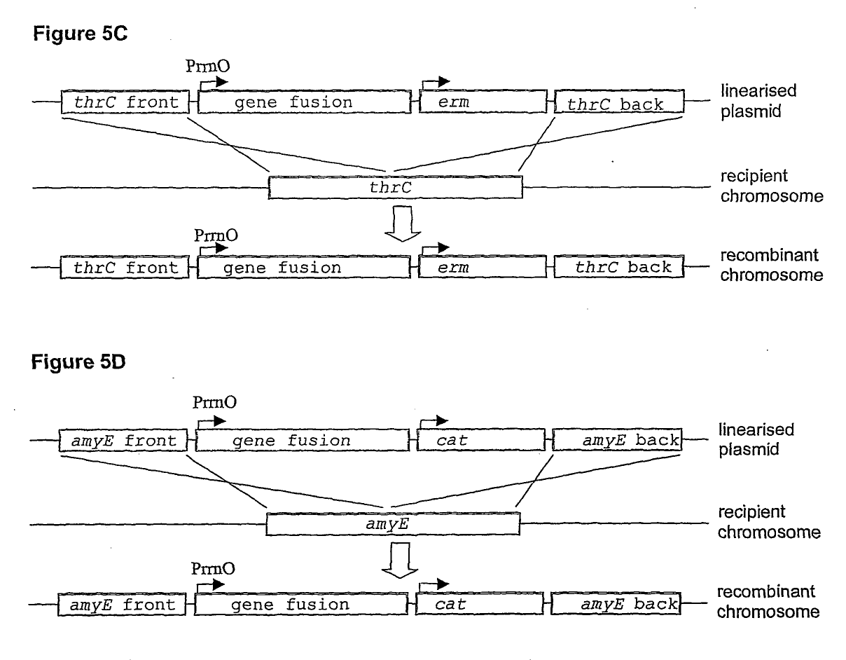

FIG. 5 describes a strategy to insert DNA/genes into the B. subtilis genome 50. The strategy uses double crossover recombination 52 using the pDL242/pDL243 vectors. More specifically the DNA of hlyA or the heterologous protein/antigen to be cloned into B. subtilis is first cloned into pDL242 or pDL243. The plasmid is then linearised and introduced into competent cells of B. subtilis by DNA mediated transformation. Since the DNA is linear it can only be introduced into the B. subtilis chromosome/genome 50 by a double crossover recombination 52 or marker replacement at either the thrC or amyE loci. Selection for recombinants is made for Cm^{R} (resistance to chloramphenicol 5 μg/ml) conferred by pDL243 and erythromycin resistance (1 μg/ml) conferred by pDL243. If the recipient species is not B. subtilis then it must be sensitive to either chloramphenicol or erythromycin to use this strategy. Such selection strategies may be used in the production of any of the Bacilli of the invention.

The pDL242 and pDL243 vectors are described in more detail in FIG. 5B, which depicts the fine detail of the site of insertion of the further heterologous protein/antigen DNA or hlyA DNA in the pDL242 or pDL243 vectors. The −35 and −10 regions of the PrrnO promoter are shown together with the ribosome binding site (Shine-Dalgarno sequence or RBS), the start of transcription (+1), the start codon (Met) and the multiple cloning site (MCS). The DNA is cloned into the MCS using PCR and primers designed to allow optimal expression.

The abbreviations used are:

thrC—the threonine C gene;

amyE—the amylase E gene;

cat—the chloramphenicol resistance gene; and

erm—the erythromycin resistance gene.

DNA in pDL242 or pDL243 is then inserted at the thrC locus on the B. subtilis chromosome, as illustrated in FIG. 5C which is a schematic representation of the marker replacement that would occur following introduction of linearised plasmid pDL242 DNA into the host B. subtilis cell. Recombination occurs between homologous segments of the thrC gene carried on the linearised plasmid vector and B. subtilis chromosome as shown.

DNA in pDL242 or pDL243 can also be inserted at the amyE locus on the B. subtilis chromosome as illustrated in FIG. 5D. Recombination occurs between homologous segments of the amyE gene carried on the linearised plasmid vector and B. subtilis chromosome as shown.

### Methods

### 1. Stable Expression of Genes in Bacilli

Two plasmid vectors, pDL243 and pDL242, are examples of vectors which can be used for integration of cloned DNA into the chromosome of Bacilli and in particular B. subtilis (FIGS. 5A-D). pDL242 is derived from pDG1664(5) and pDL243 from pDG364 (Karmazyn-Campelli, C. et al (1992) Biochimie 74:689-940). With each, the cloned DNA is introduced into a multiple cloning site of the vector. The multiple cloning site (MCS) is adjacent to the PrrnO promoter and translational start signals (ribosome binding site and ATG start codon) enabling simplified expression linked to a strong vegetatively expressed promoter (FIG. 5B). The plasmid is then linearised by cleavage (using restriction enzymes) of the plasmid backbone. The linearised DNA is then introduced into the host bacterium whereby a double crossover recombination event between homologous sequences occur. For pDL243 (FIG. 5D) this occurs at the amyE locus (amylase biosynthesis) and for pDL242 at the thrC locus (threonine biosynthesis) (FIG. 5C). In each case the plasmid vector carries upstream and downstream ends of the amyE or thrC genes enabling strand exchange. In each case recombinant organisms arising from this integration are selected using drug-resistant genes carried on the vector (Cm^{R} for pDL243 and Erm^{R} for pDL242).

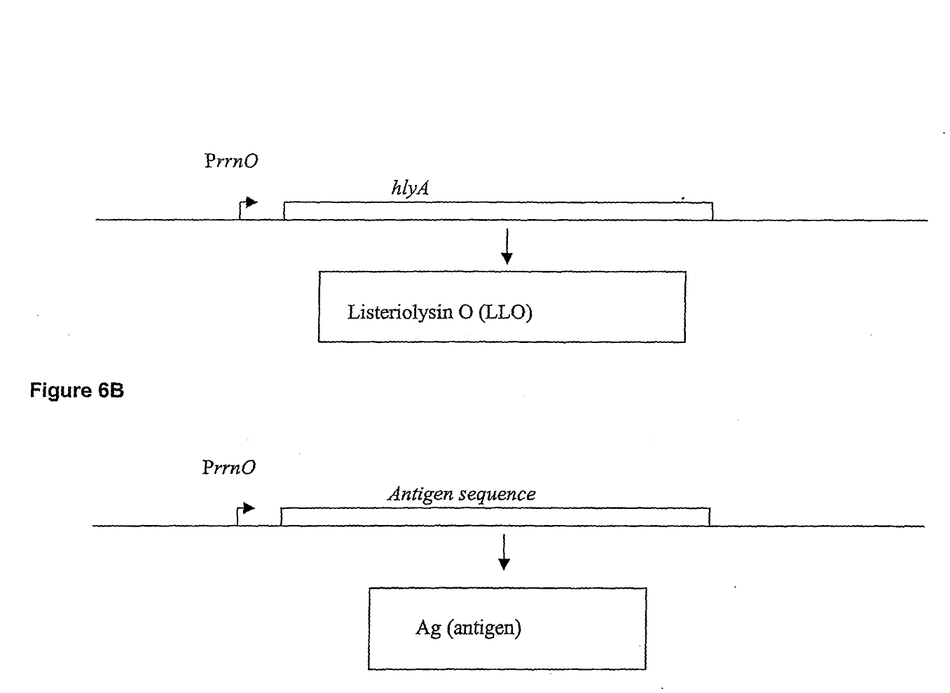

This strategy can be used to express either a further heterologous protein/antigen or LLO (see FIG. 6).

When fused to PrrnO any heterologous polypeptide gene is expressed only in the vegetative cell or in the germinated spore.

To make constructs expressing the rupturing agent and the further heterologous polypeptide in the vegetative cell/germinated spore a strain carrying PrrnO fused to one of the coding sequences may be transformed with a polynucleotide sequence comprising PrrnO fused to the other. The pDL242 or pDL243 clones may be employed or variant thereof. In a preferred instance the rupturing agent is LLO and/or the further heterologous polypeptide comprises an antigen.

### 2. Expression of a Secretable Heterologous Polypeptide in the Germinated Spore

To enable secretion of a further heterologous polypeptide a signal sequence may be present at the N-terminus of the candidate heterologous polypeptide. In B. subtilis the signal sequence is ordinarily 23-32 amino acids in length, hydrophobic and carries a recognition motif for cleavage by signal peptidase I. A signal sequence would first be fused in frame to the antigen sequence and then cloned into suitable vectors such as, for instance, pDL242 or pDL243. Any of the expressed polypeptides of the invention may comprise such a signal sequence and may comprise a cleavage signal. In a preferred instance the cleavage signal is that for signal peptidase I, a functional fragment thereof or a functional variant of either.

In a preferred instance the rupturing agent is LLO, a functional fragment there of or a functional variant of either. In one preferred instance, to enable secretion of an LLO-Antigen chimeric protein (where the antigen is the further heterologous polypeptide) the LLO (hlyA) sequence may be spliced to the antigen sequence using PCR techniques. The DNA coding the antigen may be fused to either the extreme C-terminus of LLO, or to a C-terminally deleted form of LLO, or, alternatively, inserted internally. The Ag DNA is typically not fused to the N-terminus of LLO so as not to hinder secretion of the LLO-Ag chimera.

The LLO-Ag chimeric gene sequence may be cloned into pDL242 or pDL243 such that the gene is placed under the control of PrrnO as described. This plasmid (linearised) may then be then introduced into a strain carrying either PrrnO-LLO at the amyE or thrC loci enabling creation of a strain caring PrrnO-LLO and PrrnO-LLO-Ag. A strain comprising PrrnO-LLO-Ag may also be generated. In other instances, the order of the LLO and the further heterologous polypeptide may be swapped so that either may occur first in N terminal to C terminal order in the chimeric fusion.

### 3. Expression of the Heterologous Polypeptide on the Spore Coat

Expression of a further heterologous polypeptide in the spore coat may be achieved by genetic splicing of the heterologous polypeptide to the C-termini or N-termini of the spore coat protein and in particular of the CotA, CotB, CotC, CotD, CotE, CotE coat proteins. A functional fragment or variant of such Cot proteins may be employed fused to the heterologous polypeptide. PCR may be used to splice the heterologous polypeptide DNA sequence to that of the corresponding cot gene as may restriction enzyme digests and ligation. Here, the Cot-heterologous polypeptide fusion is preferably expressed during sporulation so the chimeric gene is preferably be under the control of the natural spore coat promoter (PcotA, PcotB, PcotC, PcotD, PcotE, PcotF) or a functional fragment or variant thereof. This may be achieved by ensuring that the cot DNA that is spliced to the heterologous polypeptide DNA carries the promoter sequence. Finally the cot-antigen chimeric DNA is cloned into any suitable vector and in particular either pDG364 (Guerout-Fleury, A. M. et al (1996) Gene 180:57-61; Karmazyn-Campelli, C. et al (1992) Biochimie 74:689-94) or pDG1664, the DNA may then be linearised and then inserted into the chromsome of B. subtilis using a double crossover recombination.

Strains carrying the spore coat chimera (heterologous protein/antigen expressed on the spore coat) are then, for instance, transformed with either pDL242 or pDL243 carrying the PrrnO-LLO gene.

The following Examples illustrate the invention.

### EXAMPLE 1

### Expression of LLO in the Vegetative Cell of B. Subtilis

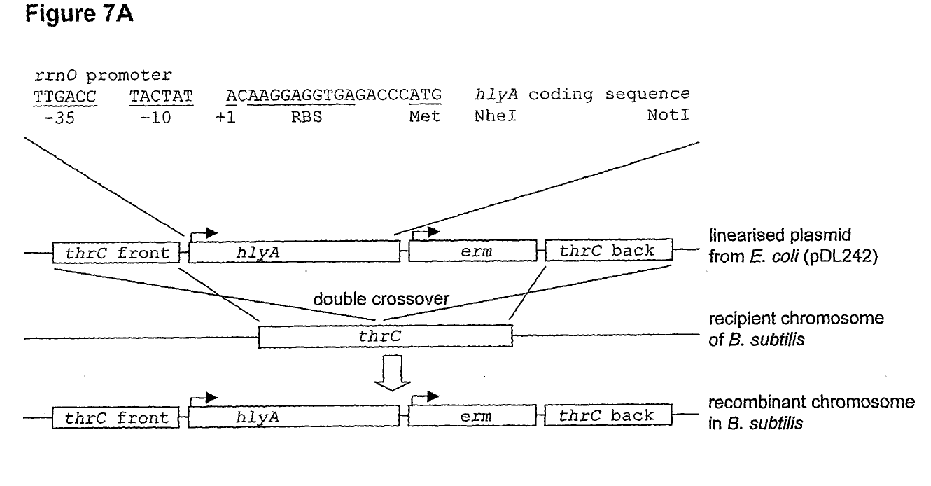

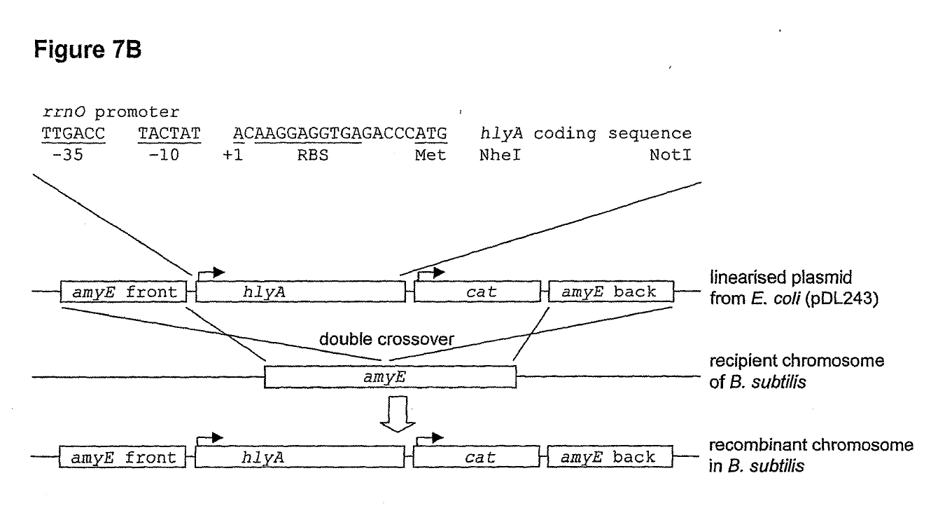

Recombinant DNA methods were used to fuse hylA sequences which encode LLO (the sequence of which is provided in FIGS. 3 and 4 and also SEQ ID Nos 1 and 2 respectively) to the PrrnO promoter and translational initiation signals carried in pDL242 (FIG. 7A) and pDL243 (FIG. 7B). In each case the PrrnO-LLO expression cassette was inserted at the thrC (FIG. 7A) and amyE (FIG. 7B) loci of B. subtilis by a double crossover recombination. Stable transformants were isolated and shown to express the 58.7 kD LLO protein during vegetative growth.

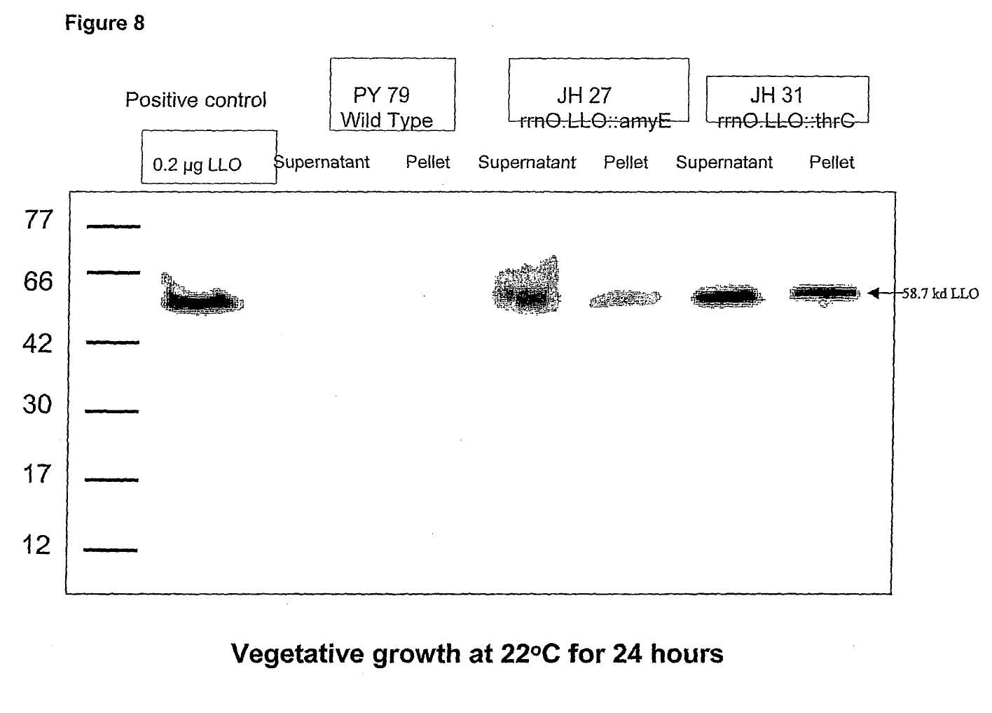

FIG. 8 shows LLO expression in cells carrying PrrnO-LLO at the thrC locus grown in LB medium and under vegetative growth. Total cells were harvested by centrifugation after 22 hours of incubation at 22° C. The cell pellet was then extracted in SDS-PAGE buffer and run on an SDS-PAGE gel and western blotted using a polyclonal antibody to detect LLO, LLO was shown to be present in the cell pellet. Culture supernatants were also examined and found to carry LLO using a polyclonal antibody to detect LLO protein in Western blots of total supernatant protein. Before analysis the supernatant was filtered through a 0.45 micron filter to remove bacteria.

These results show that LLO can be stably expressed and it is secreted from B. subtilis cells since it is found at significant levels in the culture supernatant.

### EXAMPLE 2

### Induction of CTL Responses to β-Galactosidase and Enhancement by Membrane Rupturing Agent

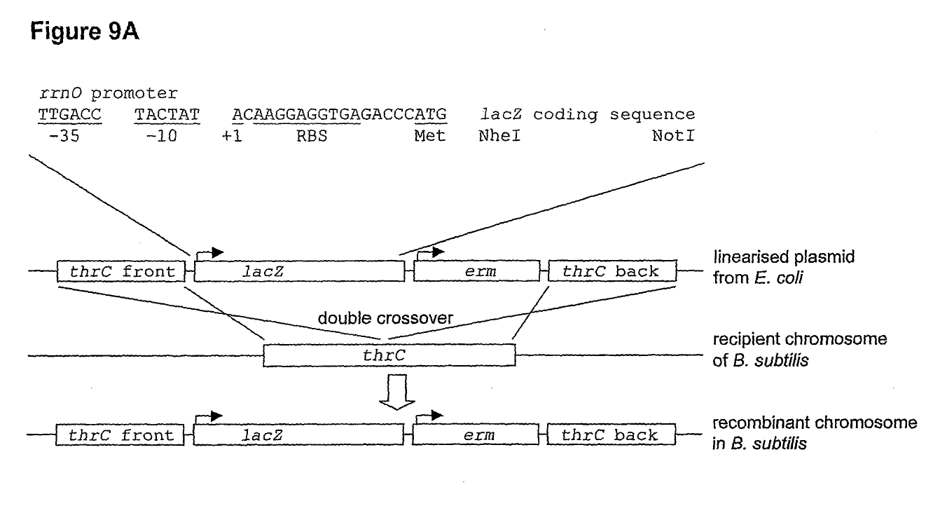

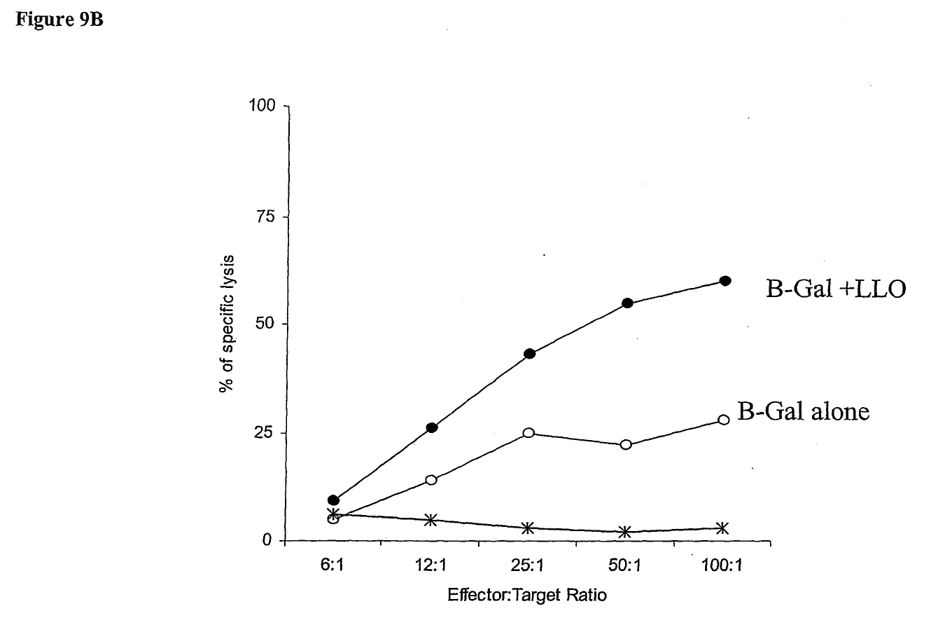

B. subtilis was engineered to carry two recombinant genes, PrrnO-LLO at the amyE locus using pDL243 (FIG. 7B) and PrrnO-lacZ at the thrC locus using pDL242 (FIG. 9A). Mice were immunized by the oral route with these B. subtilis spores (PrrnO-lacZ+PrrnO-LLO) as well as spores expressing only β-galactosidase (PrrnO-lacZ) (on days 0/1/2/20/21/22 with 2×10¹⁰ spores/dose).

Spleen cells were isolated on day 45 and maintained for 2 weeks with in vitro stimulation with the β-galactosidase dominant MHC-class I peptide (T9L). Target cell P815 was coated with T9L peptide and incubated with sodium chromate (⁵¹Cr) for 90 min, washed then lysed with different ratios of effector:target cells. Release of ⁵¹Cr was measured with a gamma counter, and data are presented as the mean value of triplicate samples (FIG. 9B: closed circle—PrrnO-lacZ and PrrnO-LLO; open circle—PrrnO-lacZ only; asterisks—naïve mice) and show CTL responses to β-galactosidase that are substantially enhanced by the co-expression of phagosome membrane ruputuring agents such as LLO in cells.

### EXAMPLE 3

### Induction of CTL Response to Influenza NP and Enhancement by Membrane Rupturing Agent

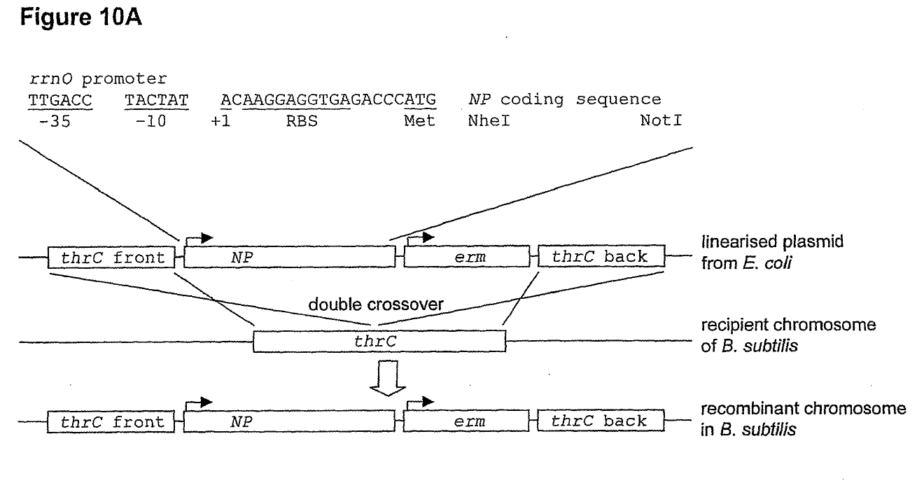

B. subtilis was engineered to express LLO and Influenza Nucleoprotein (NP) in the germinating spore or vegetative cell by fusing LLO and NP to the PrrnO-promoters. PrrnO-LLO was carred at the amyE locus using pDL243 (FIG. 7B) and PrrnO-NP at the thrC locus using pDL242 (FIG. 10A). Mice were immunized by the nasal route on days 0/1/15/16 with 2×10⁹ spores/dose route. On day 35 spleens were removed and splenocytes maintained for 2 weeks with in vitro stimulation with the peptide ELRSRYWAI (NP380-388). Target cell B-lymphoblastoid was coated with NP380-388 and incubated with sodium chromate (⁵¹Cr) for 90 min, then washed and lysed with different ratios of effector:target cells. Release of ⁵¹Cr was measured with a gamma counter. Similar levels of lysis were observed in each of five replicates (FIG. 10B-Open bar, PrrnO-LLO and PrrnO-NP; Black bar, PrrnO-NP only and Striped bar, naïve mice.).

The results show that phagosome membrane rupturing agents such as LLO enhance CTL responses to NP.

### EXAMPLE 4

### Induction and Enhancement of CTL to Influenza NP Carried on the Snore Coat and Enhancement by Membrane Rupturing Agent

Spores carrying PcotC-NP and the germinated spore PrrnO-LLO were employed. The spores were grown that carried the CotC spore coat protein fused, in frame, at its C-terminus with Influenza NP (PcotC-NP). This construct was carried at the amyE locus using pDG364 to introduce the PcotC-NP chimera. PcotC-NP was introduced at the amyE locus of cells already carrying PrrnO-LLO at the thrC locus (using pDL242). In this way spores express high levels of NP on the spore surface and when they germinate they express and deliver LLO.

The cytotoxic effect on spleen cells from mice immunised by the nasal route (as Example 3) with B. subtilis spores expressing PcotC-NP and PrrnO-LLO was determined (FIG. 11). Cells were maintained for 2 weeks in vitro stimulation with the peptide ELRSRYWAI (NP380-388), before assaying for their ability to lyse the target cells ⁵¹Cr-labelled B-lymphoblastoid coated with NP380-388.

Similar levels of lysis were observed in each of five replicates (FIG. 11-Open bar, mice receiving PrrnO-LLO and PcotC-NP; Black bar, mice receiving PcotC-NP only; striped bar, naïve mice). These results show that membrane rupturing agents such as LLO can enhance CTL responses to NP when expressed on the spore surface.

### EXAMPLE 5

### Induction and Enhancement of CTL Responses to HIV Rev

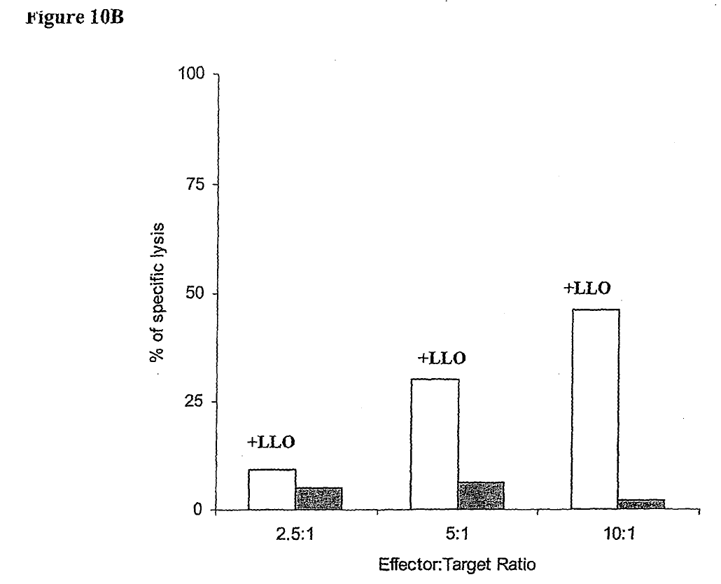

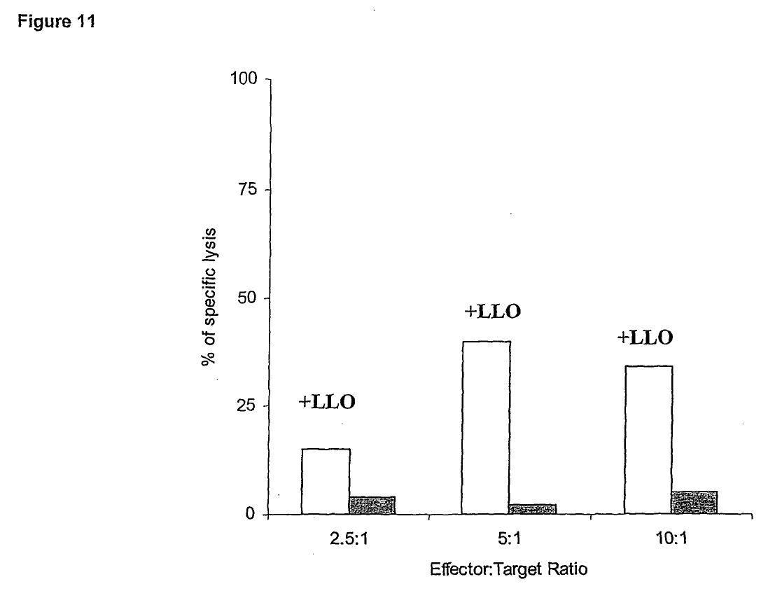

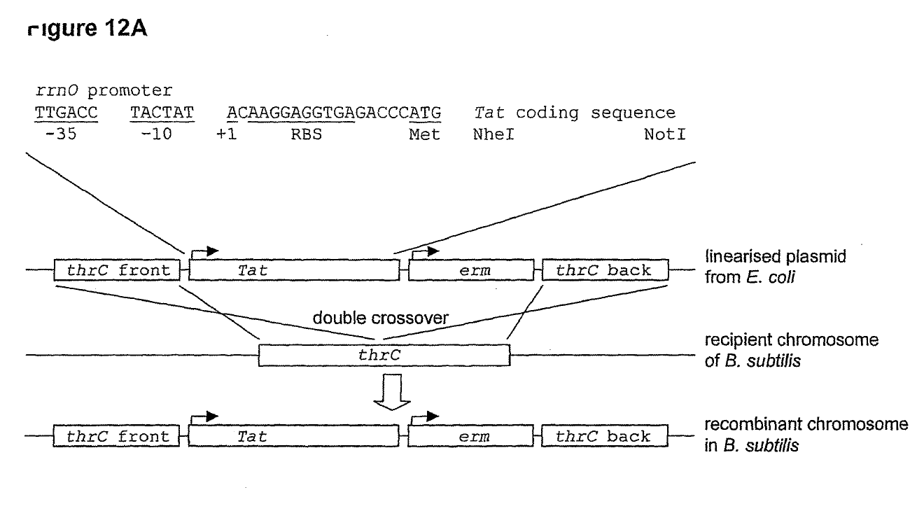

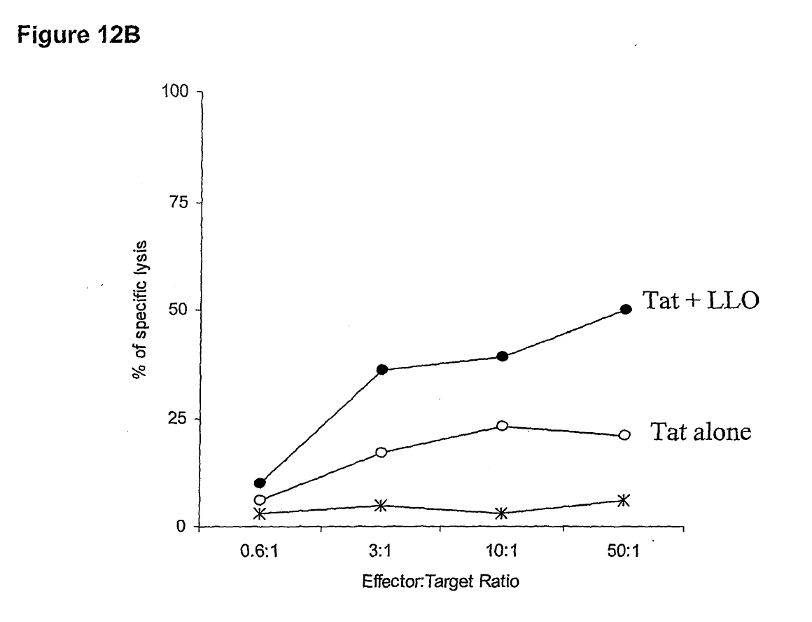

An expression cassette was constructed that expressed LLO in the germinating spore using pDL242 (FIG. 12A) and the HIV early regulatory protein, Tat, by fusing the tat gene to PrrnO using pDL243. Spores carrying this construct express LLO and Tat when the spore germinates. Mice were immunised by the intra-peritoneal route with B. subtilis carrying PrrnO-LLO and PrrnO-Tat. Spleen cells were removed 10 days postimmunisation and maintained for 10 days with in vitro stimulation with Tat peptide.

Target cells P815 were coated with Tat peptide and irradiated with ⁵¹Cr for 1 hour before being lysed with different ratios of effector:target cells. Release of ⁵¹Cr was measured with a gamma counter, and data are presented as the mean value of triplicate samples in FIG. 12B (closed circle mice receiving PrrnO-tat and PrrnO-LLO; open circle mice receiving PrrnO-tat only; asterisks—naïve mice).

The data shows evidence that CTL responses against Tat are enhanced by the action of LLO.

### EXAMPLE 6

### Proliferation of Spores/Vegetative Bacteria in Macrophages

Macrophages of the RAW264.7 cell line (intestinal macrophages) were cultured in vitro as described in L. H. Duc, H. A. Hong, N. Q. Uyen, S. M. Cutting, Vaccine 22, 1873-1885 (2004). Spores were added at a ratio of 10 spores to one macrophage in microtitre wells and incubated at 37° C. At time points thereafter (as indicated in FIG. 13) macrophages were washed and divided into two portions. One portion was heated at 68° C. for 1 hour to determine the number of spores that had been phagocytosed. The other portion was unheated and the total number of viable units (spores+vegetative cells or germinated spores) was measured.

FIG. 13 shows the CFU (colony forming units) for vegetative cells (Total counts−Spore counts). The data shows clearly that wild type spores (PY79) survive for 18 h and are rapidly cleared. By contrast spores expressing LLO (JH27 PrrnO-LLO) in vegetative cells or germinated spores are able to proliferate since the counts are much higher relative to PY79.

Therefore intracellular expression of phagosome membrane rupturing agents, such as LLO, allows survival and proliferation of germinated spores/vegetative bacteria in macrophages.

### EXAMPLE 7

### IL-1α Induction

FIG. 14 illustrates the relative levels of the IL-1α cytokine produced in RAW264.7 macrophages that have been infected (co-cultured) with spores of PY79 (control, wild type spores), JH27 (PrrnO LLO), JH95 (PrrnO-tetC) and JH49 (PrrnO-tetC PrrnO-LLO).

PrrnO-tetC is a tetanus antigen that is expressed in vegetative cells. FIG. 14 shows clearly that macrophages in which B. subtilis (JH27 or JH49) can proliferate display induced expression of the IL-1α cytokine. Expression was measured by RT-PCR analysis of IL-1α mRNA as described in L. H. Duc, H. A. Hong, N. Q. Uyen, S. M. Cutting, Vaccine 22, 1873-1885 (2004) and L. H. Duc, H. A. Hong, T. M. Barbosa, A. O. Henriques, S. M. Cutting, App. Environ. Microbiol. 70, 2161-2171 (2004).

IL-1α is a known inducer of CTL responses (Staats et al, 2001, J. Immunol. 167: 5386-5394)

### EXAMPLE 8

### IL-6 Induction

Example 8 was performed in an identical manner to Example 7, but instead of measuring IL-1α, the IL-6 cytokine was measured (FIG. 15). Again, macrophages infected with JH27 and JH49 spores, which proliferate in macrophages, showed an induction of the expression of the IL-6 cytokine.

### EXAMPLE 9

### TNF-α Induction

Example 9 was performed in an identical manner to Examples 7 and 8, but instead of measuring IL-1α or IL-6, TNF-α (Tumour necrosis factor alpha) levels were measured. No induction of this TNF-α is seen (FIG. 16), this is important because proinflammatory cytokines are not considered beneficial and could produce side effects if generated in a host.

### EXAMPLE 10

### Induction of CTL to HIV Tat

An expression cassette was constructed that expressed LLO in the germinating spore using pDL242 and the HIV early regulatory protein, Tat, by fusing the tat gene to PrrnO using pDL243. Spores carrying this construct express LLO and Tat when the spore germinates. Another construct was made where the tat gene was fused to the first 1,323 base pairs of hlyA gene. B. subtilis carrying this construct expresses an LLO-Tat fusion protein when the spores germinate, but this fusion would be defective from hemolytic activity.

Mice were immunised by the intra-peritoneal route with B. subtilis carrying PrrnO-LLO and PrrnO-Tat, or PrrnO-LLO and PrrnO-LLO-Tat. Spleen cells were removed 10 days postimmunisation and maintained for 10 days with in vitro stimulation with the tat peptide. Target cells P815 were coated with tat peptide and irradiated with ⁵¹Cr for 1 h before lysed with different ratios of effector:target cells. Release of ⁵¹Cr was measured with a gamma counter, and data are presented as the mean value of triplicate samples in FIG. 17 (open circles—naïve mice; closed circles—mice received spores carrying PrrnO-Tat only; open squares—mice received spores carrying PrrnO-Tat and PrrnO-LLO; closed squares—mice received spore carrying PrrnO-LLO-Tat and PrrnO-LLO).

The data shows evidence of CTL responses against tat enhanced by the action of LLO, and the effect was observed more clearly when tat was fused to LLO.

1-21. (canceled)

22. Non-pathogenic Bacillus spores comprising:
(i) a polynucleotide sequence encoding a phagosome membrane-rupturing agent; and
(ii) a polynucleotide sequence encoding at least one further heterologous polypeptide.

23. Non-pathogenic Bacillus spores according to claim 22, wherein the Bacillus is one of Bacillus alvei; Bacillus badius; Bacillus brevis; Bacillus cereus; Bacilluscoagulans; Bacillus fastidiosus; Bacilluslicheniformis; Bacillus jnycoides; Bacillus pasteurii; Bacillus sphaericus; Bacillus aneurinolyticus; Bacillus car otarum; Bacillus flexus; Bacillus freudenreichi; Bacillus ynaeroide; Bacillus similibedius; Bacillus thiaminolyticus; Bacillus subtilis; Bacillus pumilus; Bacillus vallismortis; Bacillusbengalicus; Bacillus flexus; and Bacillus licheniformis.

24. Non-pathogenic Bacillus spores according to claim 23, wherein the Bacillus is Bacillus subtilis.

25. Non-pathogenic Bacillus spores according to claim 22, wherein the Bacillus is a non-pathogenic Bacillus anthracis species.

26. Non-pathogenic Bacillus spores according to claim 22, wherein one or more of the heterologous polypeptide(s) of (ii) comprise an antigen, an immunogenic fragment thereof, or an immunogenic variant of either.

27. Non-pathogenic Bacillus spores according to claim 26, wherein the antigen, immunogenic fragment or immunogenic variant of either is a pathogen antigen, an autoimmune antigen, an allergic antigen, a cancer antigen or a fragment or variant of any of the preceding.

28. Non-pathogenic Bacillus spores according to claim 27 where the pathogen is a virus, bacterium, parasite, protozoan, fungus, or prion

29. Non-pathogenic Bacillus spores according to claim 28, wherein the pathogen is a virus selected from Human Papilloma Viruses (HPV), HIV, HSV2/HSV1, influenza virus (types A3 B and C), Polio virus, RSV virus, Rhinoviruses, Rotaviruses, Hepatitis A virus, Norwalk Virus Group, Enteroviruses, Astroviruses, Measles virus, Para Influenza virus, Mumps virus, Varicella-Zoster virus, Cytomegalovirus, Epstein-Barr virus, Adenoviruses, Rubella virus, Human T-cell Lymphoma type I virus (HTLV-I)5 Hepatitis B virus (HBV), Hepatitis C virus (HCV), Hepatitis D virus, Pox virus, Marburg and Ebola.

30. Non-pathogenic spores according to claim 28 wherein the antigen is a toxin antigen, immunogenic fragment thereof or an immunogenic variant of either.

31. Non-pathogenic Bacillus spores according to claim 28 where the pathogen is selected from Mycobacterium tuberculosis, Mycobacterium leprae, Listeria monocytogenes, Salmonella typhi, Shigella dysenteriae, Yersinia pestis, a Brucella species, Legionella pneumophila, Rickettsiae, Chlamydia and Bacillus anthracis.

32. Non-pathogenic Bacillus spores according to claim 22, wherein the phagosome membrane-rupturing agent is a haemolysin, a functional fragment thereof, or a functional variant of either.

33. Non-pathogenic Bacillus spores according to claim 32 wherein the haemolysin is listeriolysin O (LLO), a functional fragment thereof, or a functional variant of either.

34. A pharmaceutical composition comprising non-pathogenic Bacillus spores according to claim 22 and a pharmaceutically acceptable carrier, diluent or excipient.

35. A pharmaceutical composition according to claim 34 which is a vaccine composition.

36. A method for treating or preventing infection, autoimmunity, allergy or cancer, the method comprising administering to a human, or non-human animal, an effective amount of non-pathogenic Bacillus spores according to claim 22.

37. A method according to claim 36, wherein the method is a method of vaccination or immunisation.

38. A method of producing non-pathogenic Bacillus spores as defined in claim 22, the method comprising:
(i) transforming into vegetative cells of the Bacillus a polynucleotide sequence encoding:
(a) a phagosome membrane rupturing agent; and/or
(b) a further heterologous peptide, wherein either both are transformed into the Bacillus or the Bacillus already comprises one of the sequences of (i) or (ii); and
(ii) inducing or allowing the Bacillus to sporulate in order to produce spores.

39. Vegetative cells of a Bacillus comprising:
(i) a polynucleotide sequence encoding a phagosome membrane- rupturing agent; and
(ii) a polynucleotide sequence encoding at least one further heterologous polypeptide.


SEQUENCE LISTING (2 sequences)

SEQ ID NO: 1 - DNA, 1590 bp, Listeria monocytogenes
  CDS (1)..(1590): Coding sequence of the hyla gene encoding LLO
atgaaaaaaataatgctagtttttattacacttatattaattagtctaccaatagcacaacaaactgaagcgaaggacgcatctgcatttcataaagaagatttaatttcatccatggcaccaccaacatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatatcgttgtggagaaaaagaagaaatccatcaatcaaaataatgcagacatccaagttgtaaatgcaatttcgagcctaacatatccaggtgctctcgtaaaagcgaattcggaattagtagaaaatcaaccagatgttctccctgtaaaacgtgattcattaacacttagcatcgatttgccaggaatgactaatcaagacaataaaatcgttgtaaaaaatgctactaaatcgaatgttaacaacgcagtaaatacattagtggaaagatggaatgaaaaatatgctcaagcttatccgaatgtaagtgcaaaaattgattatgatgacgaaatggcttacagtgaatcacaattaattgcaaaatttggtactgcatttaaagctgtaaataatagtttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtaaatgcagaaaatcctcctgcatatatctcaagtgtggcatacggccgtcaagtttatttgaaattatcgactaattcccatagtactaaagtaaaagctgcttttgatgctgccgtaagtgggaaatctgtctcaggtgatgtagaattaacaaatatcatcaaaaattcttccttcaaagccgtaatttacggtggttccgcaaaagatgaagttcaaatcatcgatggcaacctcggagacttacgagatattttgaaaaaaggtgctacttttaatcgagaaacaccaggagttcccattgcttatacaacaaatttcttaaaagacaatgaattagctgttattaaaaacaactcagaatatattgaaacaacttcaaaagcttatacagatggaaaaattaatattgatcactctggaggctacgttgctcaattcaacatctcttgggatgaaataaattatgatcctgaaggtaacgaaattgttcaacataaaaactggagcgaaaacaataaaagcaagctagctcatttcacatcgtccatctatttgccaggtaacgcaagaaatattaatgtttacgccaaagaatgcactggtttagcttgggaatggtggagaacggtaattgatgaccggaacttaccacttgtgaaaaatagaaatatctccatctggggcactacgctttatccgaaatatagtaatagtgtagataatccaatcgaataa

SEQ ID NO: 2 - protein, 529 aa, Listeria monocytogenes
MKKIMLVFITLILISLPIAQQTEAKDASAFHKEDLISSMAPPTSPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEINYDPEGNEIVQHKNWSENNKSKLAHFTSSIYLPGNARNINVYAKECTGLAWEWWRTVIDDRNLPLVKNRNISIWGTTLYPKYSNSVDNPIE